(19) United States
(12) Patent Application Publication
(10) Pub. No.: US 2007/0191319 A1
(43) Pub. Date: Aug. 16, 2007

(54) Treatment of conditions that present with low bone mass by continuous combination therapy with selective prostaglandin ep4 receptor agonists and an estrogen

(75) Inventors: Hua Zhu Ke, Newbury Park, CA (US); David Duane Thompson, Gales Ferry, CT (US)

Correspondence Address: PFIZER INC., PATENT DEPARTMENT, MS8260-1611, EASTERN POINT ROAD, GROTON, CT, 06340, US

(73) Assignee: Pfizer Inc., Groton, CT (US)

(21) Appl. No.: 10/596,504

(22) PCT Filed: Dec. 6, 2004
(86) PCT No.: PCT/IB04/04049

§ 371 Date: Jun. 15, 2006

Related U.S. Application Data
(60) Provisional application No. 60/530,839, filed on Dec. 17, 2003.

(51) Int. Cl. A61K 31/426; A61K 31/56; A61K 31/565; A61K 31/4025

(52) U.S. Cl. 514/170; 514/171; 514/365; 514/424; 514/422

(57) ABSTRACT

This invention is directed to methods for treating conditions which present with low bone mass in a patient in need thereof using continuous combination therapy with a synergistically effective combination of an EP₄ receptor selective agonist or a pharmaceutically acceptable salt thereof, such as 5-(3-{2S-[3R-hydroxy-4-(3-trifluoromethyl-phenyl)-butyl]-5-oxo-pyrrolidin-1-yl}-propyl)-thiophene-2-carboxylic acid or a pharmaceutically acceptable salt thereof; and an estrogen or a pharmaceutically effective salt thereof, The present methods are useful for treating conditions that present with low bone mass including osteoporosis, osteotomy, osteoporotic fracture, childhood idiopathic bone loss, periodontitis and low bone mass and for enhancing bone healing following facial reconstruction, maxillary reconstruction or mandibular reconstruction, inducing vertebral synostosis, enhancing long bone extension, enhancing the healing rate of a bone graft or a long bone fracture or enhancing prosthetic ingrowth in a patient in need thereof.

### FIELD OF THE INVENTION

The present invention relates to methods for treating conditions which present with low bone mass in a patient using a combination of a selective prostaglandin EP₄ agonist or a pharmaceutically acceptable salt thereof and an estrogen or a pharmaceutically acceptable salt thereof. In particular, the present invention relates to methods for treating conditions which present with low bone mass, such as osteoporosis and osteoporotic fracture and the like in a patient by continuously administering a synergistically effective combination of a selective prostaglandin EP₄ agonist or a pharmaceutically acceptable salt thereof and an estrogen, or a pharmaceutically acceptable salt thereof.

### BACKGROUND OF THE INVENTION

Osteoporosis is a systemic skeletal disease, characterized by low bone mass and deterioration of bone tissue with a consequent increase in bone fragility and susceptibility to fracture. In the U.S., the condition affects more than 25 million people and causes more than 1.3 million fractures each year, including 500,000 spine, 250,000 hip and 240,000 wrist fractures annually. Hip fractures are the most serious, and are associated with a 20% excess mortality in the year following fracture, and over 50% of the survivors being incapacitated.

The elderly are at greatest risk of osteoporosis, and the problem is therefore expected to increase significantly during the next several decades with the aging of the population and by increasing longevity. The cost of managing fractures is substantial as approximately $13.8 billion dollars were spent in the U.S. in 1995 alone. Worldwide fracture incidence is forecast to increase three-fold over the next 60 years, and one study estimates that there will be 4.5 million hip fractures worldwide in 2050. The direct as well as indirect costs of fractures are therefore expected to increase correspondingly.

Although both men and women are susceptible to skeletal disorders, including osteoporosis, women are at greater risk than men. Women experience a sharp acceleration of bone loss following menopause. The recent National Osteoporosis Risk Assessment, a study of 200,160 ambulatory postmenopausal women aged 50 years or older with no previous diagnosis of osteoporosis, using World Health Organization criteria, found that 39.6% had osteopenia and 7.2% had osteoporosis (Siris, E. S. et al., JAMA 2001, 286(22), 2815-2822). In the same study, age, personal or family history of fracture, Asian or Hispanic heritage, smoking, and cortisone use were associated with significantly increased likelihood of osteoporosis; whereas higher body mass index, African American heritage, estrogen or diuretic use, exercise, and alcohol consumption significantly decreased the likelihood.

U.S. Pat. No. 6,552,067 discloses EP₄ receptor selective agonists of formula I 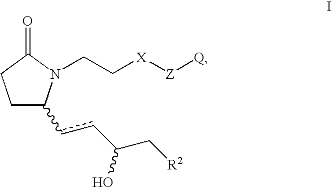
and pharmaceutical compositions comprising these compounds wherein the variables are defined as set forth therein. The compounds of formula I are useful in treating conditions which present with low bone mass, such as osteoporosis, frailty, an osteoporotic fracture, a bone defect, childhood idiopathic bone loss, alveolar bone loss, mandibular bone loss, bone fracture, osteotomy, bone loss associated with periodontitis and prosthetic ingrowth.

U.S. Patent Application Publication No. US 2002/0004495 A1 discloses methods and compositions for stimulating bone formation in a mammal using an EP₄ receptor subtype agonist optionally in combination with a bisphosphonate.

Estrogen is an agent useful for preventing and treating osteoporosis or postmenopausal bone loss in women. In addition, Black, et al., in U.S. Pat. No. 5,464,845 and EP 0605193A1 report that estrogen, particularly when taken orally, lowers plasma levels of LDL and raises those of the beneficial high density lipoproteins (HDL's). Treatment of patients with estrogen is usually referred to as hormone replacement therapy (HRT). Hormone replacement therapy has been controversial because it has been associated with increased risks for certain types of cancers.

Recently, a number of selective estrogen agonist/antagonists have been proposed for the treatment and prevention of osteoporosis. It has been reported (Osteoporosis Conference Scrip No. 1812/13 Apr. 16/20, 1993, p. 29) that raloxifene, 6-hydroxy-2-(4-hydroxyphenyl)-3-[4-(2-piperidinoethoxy)benzoyl ]benzo[b]thiophene, mimics the favorable action of estrogens on bone and lipids but, unlike estrogen, has minimal uterine stimulatory effect. Black, L. J. et al., Raloxifene (LY139481 HCl) Prevents Bone Loss and Reduces Serum Cholesterol Without Causing Uterine Hypertrophy in Ovariectomized Rats, J. Clin. Invest., 1994, 93, 63-69 and Delmas, P. D. et al., Effects of Raloxifene on Bone Mineral Density, Serum Cholesterol Concentration, and Uterine Endometrium in Postmenopausal Women, New England Journal of Medicine, 1997, 337, 1641-1647. Also, tamoxifen, 1-(4-β-dimethylaminoethoxyphenyl)-1,2-diphenyl-but-1-ene, is an antiestrogen that is proposed as an osteoporosis agent which has a palliative effect on breast cancer, but is reported to have some estrogenic activity in the uterus. U.S. Pat. No. 5,254,595 discloses agents such as droloxifene, which prevent bone loss, reduce the risk of fracture and are useful for the treatment of osteoporosis.

U.S. Pat. No. 5,552,412 discloses estrogen agonist/antagonist compounds of the formula 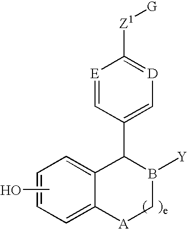
wherein the variables are defined as set forth therein. The compound (−)-cis-6-phenyl-5-(4-(2-pyrrolidin-1-yl-ethoxy)-phenyl)-5,6,7,8, -tetrahydronaphthalene-2-ol is an orally active, highly potent estrogen agonist/antagonist.

Tang et al., Restoring and Maintaining Bone in Osteogenic Female Rat Skeleton: I. Changes in Bone Mass and Structure, J. Bone Mineral Research 7 (9), p1093-1104, 1992 discloses data for the lose, restore and maintain (LRM) concept, a practical approach for reversing existing osteoporosis. The LRM concept uses anabolic agents to restore bone mass and architecture (+phase) and then switches to an agent with the established ability to maintain bone mass, to keep the new bone (+/−phase). The rat study utilized PGE₂ and risedronate, a bisphosphonate, to show that most of the new cancellous and cortical bone induced by PGE₂ can be maintained for at least 60 days after discontinuing PGE₂ by administering risedronate.

Shen et al., Effects of Reciprocal Treatment with Estrogen and Estrogen plus Parathyroid Hormone on Bone Structure and Strength in Ovariectomized Rats, J. Clinical Investigation, 1995, 96:2331-2338 discloses data for the combination and/or sequential use of anti-resorptive agents and anabolic agents for the treatment of osteoporosis.

### SUMMARY OF THE INVENTION

The present invention provides methods for treating conditions which present with low bone mass in a patient presenting with low bone mass, the method comprising continuously administering to the patient presenting with low bone mass a synergistically effective combination of an EP₄ receptor selective agonist or a pharmaceutically acceptable salt thereof and an estrogen or a pharmaceutically acceptable salt thereof. A first embodiment of the present invention is a method of treating a condition which presents with low bone mass in a patient presenting with low bone mass, the method comprising continuously administering to the patient presenting with low bone mass a synergistically effective combination of a first compound and a second compound, the first compound being of formula I 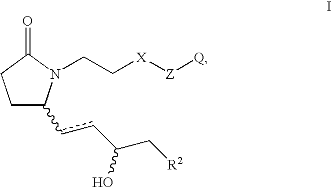
a prodrug thereof, a pharmaceutically acceptable salt of said compound or said prodrug or a stereoisomer or diastereomeric mixture of said compound, prodrug or salt, wherein:
the dotted line is a bond or no bond;
X is —CH₂— or O;
Z is —(CH₂)₃—, thienyl, thiazolyl or phenyl, provided that when X is O, then Z is phenyl;
Q is carboxyl, (C₁-C₄)alkoxylcarbonyl or tetrazolyl;
R² is —Ar or —Ar¹—V—Ar²;
V is a bond, —O—, —OCH₂— or —CH₂O—;
Ar is a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, said partially or fully saturated ring or bicyclic ring optionally having one or two oxo groups substituted on carbon or one or two oxo groups substituted on sulfur; and
Ar¹ and Ar² are each independently a partially saturated, fully saturated or fully unsaturated five to eight membered ring optionally having one to four heteroatoms selected independently from oxygen, sulfur and nitrogen, said partially or fully saturated ring optionally having one or two oxo groups substituted on carbon or one or two oxo groups substituted on sulfur;
said Ar moiety is optionally substituted on carbon or nitrogen, on one ring if the moiety is monocyclic, or on one or both rings if the moiety is bicyclic, with up to three substituents per ring each independently selected from hydroxy, halo, carboxy, (C₁-C₇)alkoxy, (C₁-C₄)alkoxy(C₁-C₄)alkyl, (C₁-C₇)alkyl, (C₂-C₇)alkenyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N-, di-N,N-, di-N,N′- or tri-N,N,N′-(C₁-C₄)alkyl substituted aminocarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl and mono-N- or di-N,N-(C₁-C₄)alkylaminosulfinyl, wherein said alkyl and alkoxy substituents in the definition of Ar are optionally substituted on carbon with up to three fluoro; and
said Ar¹ and Ar2 moieties are independently optionally substituted on carbon or nitrogen with up to three substituents each independently selected from hydroxy, halo, carboxy, (C₁-C₇)alkoxy, (C₁-C₄)alkoxy(C₁-C₄)alkyl, (C₁-C₇)alkyl, (C₂-C₇)alkenyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N-, di-N,N-, di-N,N′- or tri-N,N,N′-(C₁-C₄)alkyl substituted aminocarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl and mono-N- or di-N,N-(C₁-C₄)alkylaminosulfinyl, wherein said alkyl and alkoxy substituents in the definition of Ar¹ and Ar² are optionally substituted on carbon with up to three fluoro;
provided that (a) when X is (CH₂)— and Z is —(CH₂)₃—, then R² is not thienyl, phenyl or phenyl monosubstituted with chloro, fluoro, phenyl, methoxy, trifluoromethyl or (C₁-C₄)alkyl; and (b) when X is (CH₂)—, Z is —(CH₂)₃—, and Q is carboxyl or (C₁-C₄)alkoxycarbonyl, then R² is not (i) (C₅-C₇)cycloalkyl or (ii) phenyl, thienyl or furyl each of which may be optionally monosubstituted or disubstituted by one or two substituents selected, independently in the latter case, from halogen atoms, alkyl groups having 1-3 carbon atoms which may be substituted by one or more halogen atoms, and alkoxy groups having 1-4 carbon atoms; and the second compound is an estrogen, or a pharmaceutically acceptable salt thereof.

A second embodiment of this invention is the method of the first embodiment wherein the first compound is of the formula Ia 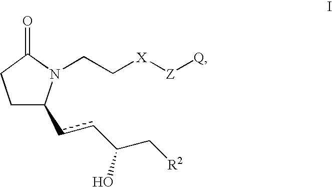
a prodrug thereof, a pharmaceutically acceptable salt of said compound or said prodrug or a stereoisomer or diastereomeric mixture of said compound, prodrug or salt, wherein: 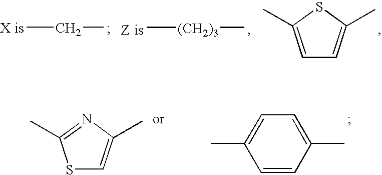
and R² is Ar wherein said Ar moiety is optionally substituted on carbon or nitrogen, on one ring if the moiety is monocyclic, or on one or both rings if the moiety is bicyclic, with up to three substituents per ring each independently selected from hydroxy, halo, carboxy, (C₁-C₇)alkoxy, (C₁-C₄)alkoxy(C₁-C₄)alkyl, (C₁-C₇)alkyl, (C₂-C₇)alkenyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C6)alkyl, (C₁-C₄)alkanoylamino, (C₁-C₄)alkoxycarbonylamino, hydroxysulfonyl, aminocarbonylamino or mono-N-, di-N,N-, di-N,N′- or tri-N,N,N′-(C₁-C₄)alkyl substituted aminocarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl and mono-N- or di-N,N-(C₁-C₄)alkylaminosulfinyl, wherein said alkyl and alkoxy substituents in the definition of Ar¹ and Ar² are optionally substituted on carbon with up to three fluoro.

A third embodiment of the present invention is the method of the second embodiment wherein the variable R² is Ar in the compound of formula Ia and Ar is cyclohexyl, 1,3-benzodioxolyl, thienyl, naphthyl or phenyl optionally substituted with one or two (C₁-C₄)alkyl, (C₁-C₄)alkoxy, (C₁-C₄)alkoxy(C₁-C₄)alkyl, chloro, fluoro, trifluoromethyl or cyano, wherein said alkyl and alkoxy substituents in the definition of Ar are optionally substituted with up to three fluoro. A fourth embodiment of this invention is the method of the third embodiment wherein the variables in the compound of formula Ia are further defined as follows: the dotted line is no bond; Q is carboxy or (C₁-C₄)alkoxylcarbonyl; and Z is thienyl. A fifth embodiment of this invention is the method of the fourth embodiment wherein the variables in the compound of formula Ia are further defined as follows: Q is carboxy and Ar is phenyl optionally substituted with one (C₁-C₄)alkyl, (C₁-C₄)alkoxy, (C₁-C₄)alkoxy(C₁-C₄)alkyl, chloro, fluoro, trifluoromethyl or cyano, wherein said alkyl and alkoxy substituents in the definition of Ar are optionally substituted with up to three fluoro. A sixth embodiment of the present invention is the method of the fifth embodiment wherein the variable Ar in the compound of formula Ia is m-trifluoromethylphenyl, m-chlorophenyl or m-trifluoromethoxyphenyl. A seventh embodiment of the present invention is the method of the sixth embodiment wherein the first compound is 5-(3-(2S-(3R-hydroxy-4-(3-trifluoromethyl-phenyl)-butyl)-5-oxo-pyrrolidin-1-yl)-propyl)-thiophene-2-carboxylic acid; 5-(3-(2S-(3R-hydroxy-4-(3-trifluoromethoxy-phenyl)-butyl)-5-oxo-pyrrolidin-1-yl)-propyl)-thiophene-2-carboxylic acid or 5-(3-(2S-(4-(3-chloro-phenyl)-3R-hydroxy-butyl)-5-oxo-pyrrolidin-1-yl)-propyl)-thiophene-2-carboxylic acid, or a pharmaceutically acceptable salt thereof. An eighth embodiment of this invention is the method of the seventh embodiment wherein the first compound is 5-(3-{2S-[3R-hydroxy-4-(3-trifluoromethyl-phenyl)-butyl]-5-oxo-pyrrolidin-1-yl}-propyl)-thiophene-2-carboxylic acid or a pharmaceutically acceptable salt thereof.

A ninth embodiment of this invention is the method of any of the first through eighth embodiments wherein the second compound is 17β-estradiol or conjugated estrogens, or a pharmaceutically acceptable salt thereof. A tenth embodiment of this invention is the method of the ninth embodiment wherein the estrogen is 17β-estradiol. An eleventh embodiment of this invention is the method of the ninth embodiment wherein the estrogen is conjugated estrogens.

A twelfth embodiment of this invention is the method of any of the first through eighth embodiments wherein the second compound is a selective estrogen agonist/iantagonist or a pharmaceutically acceptable salt thereof used in place of the estrogen, or pharmaceutically acceptable salt thereof. A thirteenth embodiment of this invention is the method of the twelfth embodiment wherein the second compound is (−)-cis-6-phenyl-5-(4(2-pyrrolidin-1-yl-ethoxy)-phenyl)5,6,7,8-tetrahydronaphthalene-2-ol, or a pharmaceutically acceptable salt thereof. A fourteenth embodiment of the present invention is the method of the thirteenth embodiment wherein the second compound is (−)-cis-6-phenyl-5-(4(2-pyrrolidin-1-yl-ethoxy)-phenyl)-5,6,7,8-tetrahydronaphthalene-2-ol, D-tartrate.

A fifteenth embodiment of this invention is the method of any of the first through fourteenth embodiments wherein osteoporosis, osteoporotic fracture, osteotomy, childhood idiopathic bone loss or periodontitis is treated or wherein bone healing following facial reconstruction, maxillary reconstruction or mandibular reconstruction is enhanced, vertebral synostosis is induced, long bone extension is enhanced, the healing rate of a bone graft or a long bone fracture is enhanced or prosthetic ingrowth is enhanced.

A further embodiment of the present invention is a kit for treating conditions which present with low bone mass in a patient presenting with low bone mass, the kit comprising a first compound and second compound as described in any of the first through fifteenth embodiments, in a first and second unit dosage form, respectively, instructions for administering the first unit dosage form and second unit dosage form to a patient suffering from a condition that present with low bone mass; and a container.

An embodiment of this invention is a kit for the treatment of a condition that presents with low bone mass, the kit comprising:

a. a compound of formula I as described hereinabove, such as 5-(3-{2S-[3R-hydroxy-4-(3-trifluoromethyl-phenyl)-butyl]-5-oxo-pyrrolidin-1-yl}-propyl)-thiophene-2-carboxylic acid or a pharmaceutically acceptable salt thereof and a pharmaceutically acceptable carrier or diluent in a first unit dosage form;

b. an estrogen or a pharmaceutically acceptable salt thereof or a selective estrogen agonistlantagonist or a pharmaceutically acceptable salt thereof and a pharmaceutically acceptable carrier or diluent in a second unit dosage form;

c. instructions for administering the first unit dosage form and second unit dosage form to a patient suffering from a condition that present with low bone mass; and

d. a container.

Another embodiment of this invention is a kit as described above wherein said first unit dosage form comprises 5-(3-{2S-[3R-hydroxy-4-(3-trifluoromethyl-phenyl)-butyl]-5-oxo-pyrrolidin-1-yl}-propyl)-thiophene-2-carboxylic acid, or a pharmaceutically acceptable salt thereof and said second unit dosage form comprises 17β-estradiol.

A further embodiment of this invention is a kit as described above wherein said first unit dosage form comprises 5-(3-{2S-[3R-hydroxy-4-(3-trifluoromethyl-phenyl)-butyl]-5-oxo-pyrrolidin-1-yl}-propyl)-thiophene-2-carboxylic acid, or a pharmaceutically acceptable salt thereof and said second unit dosage form comprises conjugated estrogens.

Yet another embodiment of this invention is a kit wherein said first unit dosage form comprises 5-(3-{2S-[3R-hydroxy-4-(3-trifluoromethyl-phenyl)-butyl]-5-oxo-pyrrolidin-1-ylpropyl)-thiophene-2-carboxylic acid, or a pharmaceutically acceptable salt thereof and said second unit dosage form comprises (−)-cis-6-phenyl-5-(4-(2-pyrrolidin-1-yl-ethoxy)-phenyl)-5,6,7,8-tetrahydronaphthalene-2-ol, D-tartrate.

The methods of this invention result in higher magnitude bone mass gain than is achievable with the same doses of an EP₄ receptor selective agonist, such as 5-(3-{2S-[3R-hyd roxy-4-(3-trifluoromethyl-phenyl)-butyl]-5-oxo-pyrrolidin-1-yl}-propyl)-thiophene-2-carboxylic acid as described above, alone, or an estrogen, as described above, alone. Thus, the methods and of this invention are synergistically effective as they increase bone mass and will decrease fracture rates to a greater extent than is achievable through use of either agent alone. This invention makes a significant contribution to the art by providing methods that increase and maintain bone mass resulting in prevention, retardation, and/or regression of osteoporosis and related bone disorders. Other features and advantages will be apparent from the specification and claims that describe the invention.

### DETAILED DESCRIPTION OF THE INVENTION

The present invention is directed to methods for treating conditions which present with low bone mass in a patient presenting with low bone mass, the method comprising continuously administering to the patient presenting with low bone mass a synergistically effective combination of a first compound and a second compound, the first compound being of the formula I 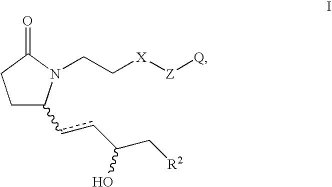
a prodrug thereof, a pharmaceutically acceptable salt of said compound or said prodrug or a stereoisomer or diastereomeric mixture of said compound, prodrug or salt, wherein the dotted line, R², X, Z and Q are as defined hereinabove; and the second compound is an estrogen, or a pharmaceutically acceptable salt thereof.

A second embodiment of this invention, is the method of the first embodiment wherein the first compound is of the formula Ia 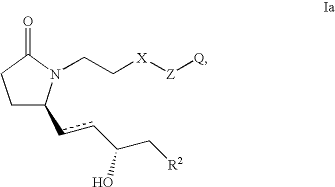
a prodrug thereof, a pharmaceutically acceptable salt of said compound or said prodrug or a stereoisomer or diastereomeric mixture of said compound, prodrug or salt, wherein X, Z and R² are defined as described hereinabove. Further non-limiting examples of embodiments of this invention are the third through fifteenth embodiments as described hereinabove.

The compounds of formulae I and la, including 5-(3-{2S-[3R-hydroxy-4-(3-trifluoromethyl-phenyl)-butyl]-5-oxo-pyrrolidin-1-yl}-propyl)-thiophene-2-carboxylic acid and the pharmaceutically acceptable salts thereof are prepared as described in U.S. Pat. No. 6,552,067. Particularly, 5-(3-{2S-[3R-hydroxy-4-(3-trifluoromethyl-phenyl)-butyl]-5-oxo-pyrrolidin-1-yl}-propyl)-thiophene-2-carboxylic acid is prepared according to the procedure as described for Example 3M in U.S. Pat. No. 6,552,067 as described hereinbelow.

The second compound used in the methods of this invention is an estrogen, or a pharmaceutically acceptable salt thereof. The second compound used in the methods of this invention can also be an estrogen agonist/antagonist, or a pharmaceutically acceptable salt thereof.

Estrogens useful in the methods of this invention include estrone, estriol, equilin, estradiene, equilenin, ethinyl estradiol, 17β-estradiol, 17α-dihydroequilenin, 17β-dihydroequilenin (U.S. Pat. No. 2,834,712), 17α-dihydroequilin, 17β-dihydroequilin and menstranol. Phytoestrogens, such as equol or enterolactone, may also be used in the present compositions, methods and kits. Esterified estrogens, such as those sold by Solvay Pharmaceuticals, Inc. under the Estratab® tradename, may also be used in the present methods. Also useful in the present invention are the salts of the applicable estrogens, including the sodium salts. Examples of these salts are sodium estrone sulfate, sodium equilin sulfate, sodium 17α-dihydroequilin sulfate, sodium 17α-estradiol sulfate, sodium δ-8,9-dehydroestrone sulfate, sodium equilenin sulfate, sodium 17β-estradiol sulfate, sodium 17β-dihydroequilenin sulfate, estrone 3-sodium sulfate, equilin 3-sodium sulfate, 17α-dihydroequilin 3-sodium sulfate, 3β-Hydroxy-estra-5(10),7-dien-17-one 3-sodium sulfate, 5α-pregnan-3β-20R-diol 20-sodium sulfate, 5α-pregnan-3β,16α-diol-20-one 3-sodium sulfate, δ(8,9)-dehydroestrone 3-sodium sulfate, estra-3β, 17α-diol 3-sodium sulfate, 3β-Hydroxy-estr-5(10)-en-17-one 3-sodium sulfate or 5α-pregnan-3β, 16α,20R-triol 3-sodium sulfate. Salts of estrone include, but are not limited to, the sodium and piperate salts. Conjugated estrogenic hormones, such as those in Wyeth-Ayerst Laboratories' Premarin® products, referred to herein as conjugated estrogens, are also useful in the compositions, methods and kits of this invention. Although the term “conjugated estrogens” is plural it is intended to be useful as “an estrogen” and a “second compound” in the methods and kits of this invention.

In the methods of the present invention where an estrogen is employed as the second compound, the estrogen is optionally administered along with a progestin. Progestins are familiar to those skilled in the art. Examples of specific progestins that can be used in the methods of the present invention include, but are not limited to, levonorgestrel, norethindrone, ethynodiol, desogestrel, norgestrel, norgestimate, and medroxyprogesterone. It is common to use a pharmaceutically acceptable salt of the progestins, which salts are described below.

The second compound used in the methods of this invention can also be an estrogen agonistlantagonist. An “estrogen agonist/antagonist” is a compound that affects some of the same receptors that estrogen does, but not all, and in some instances, it acts as an agonist and in other instances it antagonizes or blocks estrogen. It is also known as a “selective estrogen receptor modulator” (SERM). Estrogen agonists/antagonists may also be referred to as antiestrogens although they have some estrogenic activity at some estrogen receptors. Estrogen agonists/antagonists are therefore not what are commonly referred to as “pure antiestrogens”. Antiestrogens that can also act as agonists are referred to as Type I antiestrogens. Type I antiestrogens activate the estrogen receptor to bind tightly in the nucleus for a prolonged time but with impaired receptor replenishment (Clark, et al., Steroids 1973;22:707, Capony et al., Mol Cell Endocrinol, 1975;3:233).

Estrogen agonists/antagonists useful in the methods and kits of the present invention include the compounds described in U.S. Pat. No. 5,552,412. Those compounds are described by formula (I) given below: 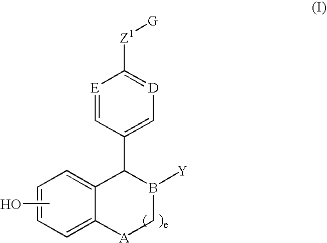
wherein the variables are as defined therein.

Additional compounds useful in the methods of this invention also disclosed in U.S. Pat. No. 5,552,412 are of the formula (IA): 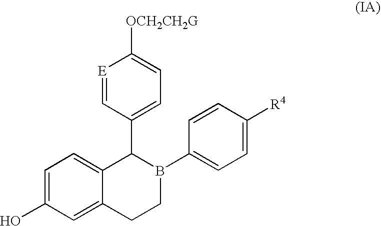
wherein the variables are defined as set forth therein.

Particular compounds useful in the methods of this invention are:

cis-6-(4-fluoro-phenyl)-5-[4-(2-piperidin-1-yl-ethoxy)-phenyl]-5,6,7,8-tetrahydro-naphthalene-2-ol;

(−)-cis-6-phenyl-5-[4-(2-pyrrolidin-1-yl-ethoxy)-phenyl]-5,6,7 ,8-tetrahydro-naphthalene-2-ol;

cis-6-phenyl-5-[4-(2-pyrrolidin-1-yl-ethoxy)-phenyl]-5,6,7,8-tetrahydro-naphthalene-2-ol;

cis-1-[6′-pyrrolidinoethoxy-3′-pyridyl]-2-phenyl-6-hydroxy-1,2,3,4-tetrahydronaphthalene;

1-(4′-pyrrolidinoethoxyphenyl)-2-(4″-fluorophenyl)-6-hydroxy-1,2,3,4-tetrahydroisoquinoline;

cis-6-(4-hydroxyphenyl)-5-[4-(2-piperidin-1-yl-ethoxy)-phenyl]-5,6,7,8-tetrahydro-naphthalene-2-ol; and

1-(4′-pyrrolidinoethoxyphenyl)-2-phenyl-6-hydroxy-1,2,3,4-tetrahydroisoquinoline and pharmaceutically acceptable salts thereof. A particular salt of (−)-cis-6-phenyl-5-[4-(2-pyrrolidin-1-yl-ethoxy)-phenyl]-5,6,7,8-tetrahydro-naphthalene-2-ol is the tartrate salt.

Other estrogen agonists/antagonists useful in the methods of this invention are disclosed in U.S. Pat. No. 5,047,431. The structure of these compounds is given by formula (II) below: 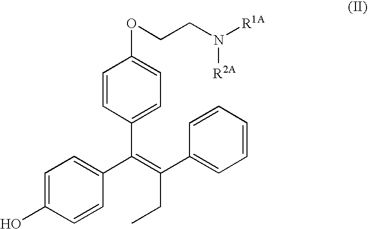
wherein R^{1A} and R^{2A} may be the same or different and are either H, methyl, ethyl or a benzyl group; and optical or geometric isomers thereof; and pharmaceutically acceptable salts, N-oxides, esters, quatemary ammonium salts, and prodrugs thereof.

Additional estrogen agonists/antagonists useful in the methods of this invention are tamoxifen: (ethanamine, 2-[-4-(1,2-diphenyl-1-butenyl)phenoxy]-N,N-dimethyl, (Z)-2-, 2-hydroxy-1,2,3-propanetricarboxylate(1:1)) and other compounds as disclosed in U.S. Pat. No. 4,536,516; 4-hydroxy tamoxifen (i.e., tamoxifen wherein the 2-phenyl moiety has a hydroxy group at the 4 position) and other compounds as disclosed in U.S. Pat. No. 4,623,660; raloxifene: (methanone, [6-hydroxy-2-(4-hydroxyphenyl)benzo[b]thien-3-yl][4-[2-(1-piperidinyl)ethoxy]phenyl]-hydrochloride) and other compounds as disclosed in U.S. Pat. Nos. 4,418,068; 5,393,763; 5,457,117; 5,478,847 and 5,641,790; toremifene: (ethanamine, 2-[4-(4-chloro-1,2-diphenyl-1-butenyl)phenoxy]-N,N-dimethyl-, (Z)-, 2-hydroxy-1,2,3-propanetricarboxylate (1:1) and other compounds as disclosed in U.S. Pat. Nos. 4,696,949 and 4,996,225; centchroman: 1-[2-[[4-(-methoxy-2,2, dimethyl-3-phenyl-chroman-4-yl)-phenoxy]-ethyl]-pyrrolidine and other compounds as disclosed in U.S. Pat. No. 3,822,287; idoxifene: pyrrolidine, 1-[-[4-[[1-(4-iodophenyl)-2-phenyl-1-butenyl]phenoxy]ethyl] and other compounds as disclosed in U.S. Pat. No. 4,839,155; 6-(4-hydroxy-phenyl)-5-[4-(2-piperidin-1-yl-ethoxy)-benzyl]-naphthalen -2-ol and other compounds as disclosed in U.S. Pat. No. 5,484,795; and {4-[2-(2-aza-bicyclo[2.2.1]hept-2-yl)-ethoxy]-phenyl}-[6-hydroxy-2-(4-hydroxy-phenyl)-benzo[b]thiophen-3-yl]-methanone and other compounds as disclosed in published international patent application WO 95/10513. Other preferred compounds include GW 5638 and GW 7604, the synthesis of which is described in Willson et al., J. Med. Chem., 1994;37:1550-1552.

Additional estrogen agonists/antagonists useful in the methods of this invention include EM-652 (as shown in formula (III) and EM-800 (as shown in formula (IV)). The synthesis of EM-652 and EM-800 and the activity of various enantiomers is described in Gauthier et al., J. Med. Chem., 1997;40:2117-2122.

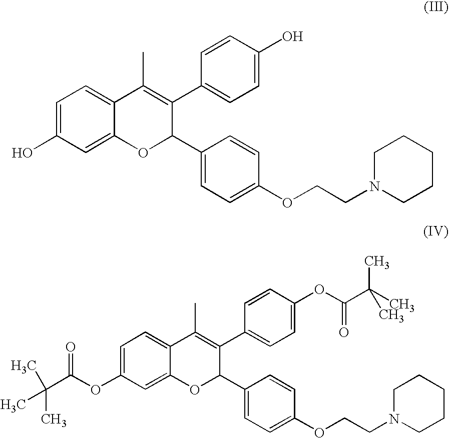

Further estrogen agonists/antagonists that can be used in the methods of this invention include TSE-424 and other compounds disclosed in U.S. Pat. No. 5,998,402, U.S. Pat. No. 5,985,910, U.S. Pat. No. 5,780,497, U.S. Pat. No. 5,880,137, and European Patent Application EP 0802183 A1 including the compounds of the formulas V and VI, below: 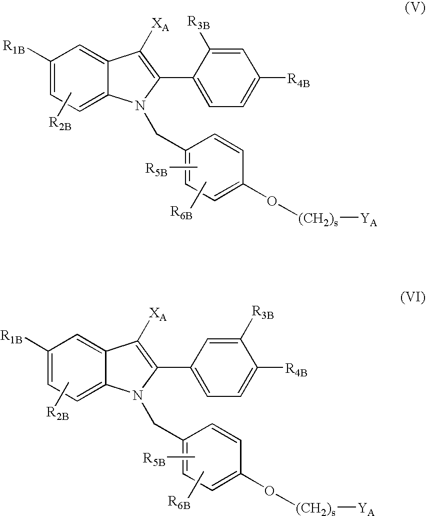
wherein the variables are defined as set forth therein. A particular estrogen agonist/antagonist useful in the methods of this invention is the compound, TSE-424, of formula (Va) below:

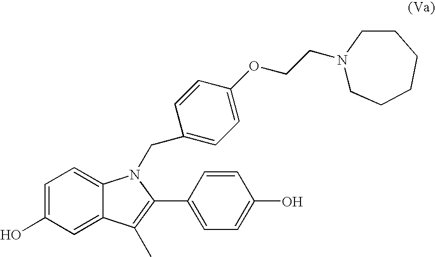

In all of the methods of this invention, it is preferred that the patient is a mammal such as a human or a companion animal. The term “companion animal” refers to a household pet or other domesticated animal such as, but not limited to, cattle, sheep, ferrets, swine, horses, poultry, fish, rabbits, goats, dogs, cats and the like. Particularly preferred companion animals are dogs and cats. In all of the methods and kits of this invention, it is particularly preferred that the mammal is a human.

The phrase “condition which presents with low bone mass” refers to a condition where the level of bone mass is below the age specific normal as defined in standards by the World Health Organization “Assessment of Fracture Risk and its Application to Screening for Postmenopausal Osteoporosis (1994), Report of a World Health Organization Study Group. World Health Organization Technical Series 843”. Childhood idiopathic and primary osteoporosis are also included. Included in the treatment of osteoporosis is the prevention or attenuation of long term complications such as curvature of the spine, loss of height, prosthetic surgery, and prevention of prostate malfunctioning. Also included is increasing the bone fracture healing rate and enhancing the rate of successful bone grafts. Also included is periodontal disease and alveolar bone loss. Specific conditions included within the definition of this phrase are osteoporosis, osteotomy, childhood idiopathic bone loss, periodontitis, bone healing following facial reconstruction, maxillary reconstruction, mandibular reconstruction and bone fracture. Further, “conditions which present with low bone mass” encompasses such conditions as interfaces between newly attached prostheses and bone that require bone ingrowth.

The phrase “condition which presents with low bone mass” also refers to a mammal known to have a significantly higher than average chance of developing such diseases as are described above including osteoporosis (e.g., post-menopausal women, men over the age of 60, and persons being treated with drugs known to cause osteoporosis as a side effect (such as certain glucocorticoids)).

Those skilled in the art will recognize that the term bone mass actually refers to bone mass per unit area that is sometimes (although not strictly correctly) referred to as bone mineral density.

The term “treating”, “treat” or “treatment” as used herein includes curative, preventative (e.g., prophylactic) and palliative treatment.

The parenthetical negative or positive sign used herein in the nomenclature denotes the direction plane polarized light is rotated by the particular stereoisomer.

When the compounds and pharmaceutically acceptable salts thereof used in the methods and kits of this invention form hydrates or solvates, such hydrates or solvates are also within the scope of the invention.

The methods and kits of this invention are all adapted to therapeutic use to either activate bone turnover or prevent bone resorption or increase bone formation in mammals, particularly humans. Since these functions are closely related to the development of osteoporosis and bone related disorders, these methods and kits, by virtue of their action on bone, prevent, arrest, regress or reverse osteoporosis.

The utility of the methods and kits of the present invention for the treatment of conditions which present with low bone mass, including osteoporosis, in mammals (e.g. humans) is demonstrated by the activity of the compounds used in the methods and kits of this invention in conventional assays as set forth in U.S. Pat. No. 5,552,412 and U.S. Pat. No. 6,552,067. Further evidence of the utility of the instant methods and kits is set forth in Example One below. Such protocols also provide a means whereby the activities of the compounds used in the methods and kits of this invention can be compared between themselves and with the activities of other known compounds. The results of these comparisons are useful for determining dosage levels in mammals, including humans, for the treatment of such diseases.

Administration of the compounds used in the methods of this invention can be via any method that delivers a compound used in the methods of this invention systemically and/or locally in a continuous fashion. These methods include oral routes, parenteral, intraduodenal and transdermal routes, etc. The compounds used in the methods and kits of this invention are administered to the patient in need thereof by continuous administration orally, parenterally (e.g., intravenous, intramuscular, transcutaneous, subcutaneous or intramedullary) or transdermally. The two different compounds used in the methods and kits of this invention can be co-administered simultaneously or sequentially in any order, or a single pharmaceutical composition comprising a first compound as described above and a second compound as described above in a pharmaceutically acceptable carrier or diluent can be administered. In a particular embodiment of this invention the first compound and second compound are administered substantially simultaneously.

In any event the amount and timing of compounds administered will, of course, be dependent on the subject being treated, on the severity of the affliction, on the manner of administration and on the judgment of the prescribing physician. Thus, because of patient to patient variability, the dosages given below are a guideline and the physician may titrate doses of the drug to achieve the activity (e.g., bone mass augmentation) that the physician considers appropriate for the individual patient. In considering the degree of activity desired, the physician must balance a variety of factors such as bone mass starting level, age of the patient, presence of preexisting disease, as well as presence of other diseases (e.g., cardiovascular). For example, the administration of (−)-cis-6-phenyl-5-(4-(2-pyrrolidin-1-yl-ethoxy)-phenyl)-5,6,7,8-tetrahydronaphthalene-2-ol can provide cardiovascular benefits, particularly for post-menopausal women. The following paragraphs provide preferred dosage ranges for the various components of this invention.

An effective dosage for an EP₄ receptor selective agonist, such as 5-(3-{2S-[3R-hydroxy-4-(3-trifluoromethyl-phenyl)-butyl}-5-oxo-pyrrolidin-1-yl}-propyl)-thiophene-2-carboxylic acid and the pharmaceutically acceptable salts thereof is about 0.001 to about 100 mg/kg/day.

An effective dosage for an estrogen, conjugated estrogens or an estrogen agonist/antagonist is in the range of about 0.0001 to about 100 mg/kg/day, particularly about 0.001 to about 10 mg/kg/day. For example, an effective dosage for 17β-estradiol or (−)-cis-6-phenyl-5-[4-(2-pyrrolidin-1-yl-ethoxy)-phenyl]-5,6,7,8-tetrahydro-naphthalene-2-ol is in the range of 0.0001 to 100 mg/kg/day, particularly 0.001 to 10 mg/kg/day.

A particular synergistically effective dosage for administration of the first compound, such as 5-(3-{2S-[3R-hydroxy-4-(3-trifluoromethyl-phenyl)-butyl}-5-oxo-pyrrolidin-1-yl}-propyl)-thiophene-2-carboxylic acid, and the second compound, such as 17β-estradiol, is about 0.3 mg/kg/day and 0.01 mg/kg/day, respectively.

Where a pharmaceutically acceptable salt of either of the first or second compounds is used in this invention, the skilled person will be able to calculate effective dosage amounts by calculating the molecular weight of the salt form and performing simple stoichiometric ratios.

The compounds used in the methods of the present invention are generally administered in the form of a pharmaceutical composition comprising at least one of the compounds or pharmaceutically acceptable salts thereof useful in this invention together with a pharmaceutically acceptable carrier or diluent. Thus, the compounds and pharmaceutically acceptable salts thereof used in the methods and kits of this invention can be administered separately or together in any conventional oral, parenteral or transdermal dosage form. When administered separately, the administration of the other compound or pharmaceutically acceptable salt thereof of the invention follows.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds or pharmaceutically acceptable salts thereof of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g.,topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of each active ingredient are known, or will be apparent in light of this disclosure, to those skilled in this art. For examples, see Remington's Pharmaceutical Sciences, Mack Publishing Company, Easton, Pa., 19th Edition (1995).

Pharmaceutical compositions according to the invention may contain 0.1%-95% of a combination of the compounds or pharmaceutically acceptable salts thereof of this invention, preferably 1%-70%. In any event, the composition or formulation to be administered will contain a quantity of the compounds or pharmaceutically acceptable salts thereof of the invention in an amount synergistically effective to treat the disease/condition of the subject being treated.

An EP₄ receptor selective agonist of formula I or estrogen or estrogen agonist/antagonist, or the pharmaceutically acceptable salts thereof, or a combination thereof can be administered in a continuous fashion in accordance with the methods of the present invention using a sustained release formulation. For purposes of discussion, not limitation, the many embodiments hereunder can be grouped into classes according to design and principle of operation.

The first class of sustained release dosage forms described below is matrix systems, which include but are not limited to 1) non-eroding matrices, tablets, multiparticulates, and hydrogel-based systems; 2) hydrophilic eroding, dispersible or dissolvable matrix systems, tablets and multiparticulates; and 3) coated matrix systems. The second class comprises reservoir systems where release of the active compound is modulated by a membrane, such as capsules, and coated tablets or multiparticulates. The third class comprises osmotic-based systems such as 1) coated bilayer tablets; 2) coated homogeneous tablet cores; 3) coated multiparticulates; and 4) osmotic capsules. The fourth class comprises swellable systems where active compound is released by swelling and extrusion of the core components out through a passageway in a coating or surrounding shell or outer layer.

A first class includes matrix systems, in which an EP₄ receptor selective agonist of formula I or estrogen or estrogen agonist/antagonist or a combination thereof (hereinafter referred to as the active component) is dissolved, embedded or dispersed in a matrix of another material that serves to retard the release of the active component into an aqueous environment [e.g., the lumenal fluid of the gastrointestinal tract (GI)]. When the active component is dissolved, embedded or dispersed in a matrix of this sort, release of the active component takes place principally from the surface of the matrix. Thus, the active component is released from the surface of a device which incorporates the matrix after it diffuses through the matrix into the surrounding fluid or when the surface of the device dissolves or erodes, exposing the active component. In some embodiments, both mechanisms can operate simultaneously. The matrix systems may be large, i.e., tablet sized (about 1 cm), or small (<0.3 cm). The system may be unitary, it may be divided by virtue of being composed of several sub-units (for example, several tablets which constitute a single dose) which are administered substantially simultaneously, it may consist of several small tablets within a capsule, or it may comprise a plurality of particles, referred to herein as a multiparticulate. A multiparticulate can have numerous formulation applications. For example, a multiparticulate may be used as small beads or as powder for filling a capsule shell, it may be compressed into a tablet, or it may be used per se for mixing with food (for example, ice cream) to increase palatability, or as a sachet that may be dispersed in a liquid, such as fruit juice or water.

The multiplicity of variables affecting release of the active component from matrix devices permits abundant flexibility in the design of devices of different materials, sizes, and release times.

Non-eroding matrix tablets that provide sustained release of the active component can be made with an active component and water insoluble materials such as waxes, cellulose, or other water insoluble polymers. Matrix materials useful for the manufacture of these dosage forms include microcrystalline cellulose such as Avicel® (FMC Corp., Philadelphia, Pa.), including grades of microcrystalline cellulose to which binders such as hydroxypropyl methyl cellulose have been added, waxes such as paraffin, modified vegetable oils, carnauba wax, hydrogenated castor oil, beeswax, and the like, as well as polymers such as cellulose, cellulose esters, cellulose ethers, poly(vinyl chloride), poly(vinyl acetate), copolymers of vinyl acetate and ethylene, polystyrene, and the like. Water soluble binders or release modifying agents which can optionally be formulated into the matrix include water-soluble polymers such as hydroxypropyl cellulose (HPC), hydroxypropyl methyl cellulose (HPMC), methyl cellulose, poly (N-vinyl-2-pyrrolidinone) (PVP), poly(ethylene oxide) (PEO), poly(vinyl alcohol) (PVA), xanthan gum, carrageenan, and other such natural and synthetic materials. In addition, materials that function as release-modifying agents include water-soluble materials such as sugars or salts. Preferred water-soluble materials include lactose, sucrose, glucose, and mannitol, as well as HPC, HPMC, and PVP. In addition, solubilizing acid excipients such as organic acids including but not limited to malic acid, citric acid, erythorbic acid, ascorbic acid, adipic acid, glutamic acid, maleic acid, aconitic acid, fumaric acid, succinic acid, tartaric acid, and aspartic acid and solubilizing excipients such as sodium bitartrate and cyclodextrins, can be incorporated into matrix tablets to increase the release rate of the active component, increase the total quantity of the active component released, and potentially increase absorption and consequently the bioavailability of the active component, particularly from matrix formulations that release the active component over a period of six hours or longer.

In addition to components of the matrix system, the size of the matrix system can affect the rate of active component release; therefore, a large matrix system such as a tablet will, in general, have a different composition from a small one such as a multiparticulate to achieve similar release profiles. The effect of the size of the matrix system on the kinetics of active component release follows scaling behavior well known to those skilled in the art. By way of illustration, the following table shows the diffusion coefficient of a active component through the matrix required to achieve a characteristic time for release of 10 hours for matrix systems of different sizes that release an active component by a diffusive-based mechanism (rather than an eroding or in combination with an eroding mechanism).

| radius (cm) | diffusion coefficient (cm²/sec) |
|---|---|
| 0.0025 (50 μm diameter) | 1.7 × 10⁻¹⁰ |
| 0.1 (2 mm diameter) | 3 × 10⁻⁷ |
| 0.5 (1 cm diameter) | 7 × 10⁻⁶ |

The above table illustrates that diffusion coefficients necessary to achieve the target characteristic time of release can change by orders of magnitude as the desired size of the device changes. Matrix materials that can be used to provide an active component diffusion coefficient at the low end of the diffusion coefficient scale are polymers such as cellulose acetate. Conversely, materials at the upper end of the scale are materials such as polymers that form hydrogels or a water-swollen mass when hydrated. The rate of diffusion for any particular device can accordingly be tailored by the material or materials selected and the structure of the matrix.

For purposes of further illustration, to obtain a sustained release non-eroding matrix in a particle of about 50 μm in diameter, a matrix material of a polymer such as cellulose acetate or a similar material will likely be required, the slow diffusing matrix material tending to offset the short distances characteristic of small particle size. In contrast, in order to obtain sustained release in a large (e.g., 1 cm) device, a material which is more liquid-like (e.g., a hydrogel or water-soluble polymer) or with greater porosity will likely be required. For devices of an intermediate size, e.g., about 1 mm in diameter, a matrix composition of intermediate characteristics can be employed.

It is also noted that the effective diffusion coefficient of an active component in a matrix may be increased to the desired value by the addition of plasticizers, pores, or pore-inducing additives, as known in the art. Slowly hydrating materials may also be used to effectively reduce the diffusion rates of an active component, particularly at times shortly after administration. In addition to changing the effective diffusion coefficient, the release rate can also be altered by the inclusion of more soluble salt forms of the active component (relative to the free acid or free base form) or excipients such as acids or bases that solubilize the active component.

A further sustained release non-eroding matrix system comprises an active component dispersed in a hydrogel matrix. This embodiment differs from the hydrophilic matrix tablet in that the hydrogel of this embodiment is not a compressed tablet of soluble or erodible granular material, but rather is a monolithic polymer network. As is known in the art, a hydrogel is a water-swellable network polymer. Hydrogels can be made in various geometries, such as caplets, tablets, and multiparticulates. As an example, tablets can be prepared by standard techniques containing 10 to 80% of a crosslinkable polymer. Once tablets are formed the polymer can be crosslinked via a chemical crosslinking agent such as gluteraldehyde or via UV irradiation forming a hydrogel matrix. Hydrogels are preferred materials for matrix devices because they can absorb or be made to contain a large volume fraction of water, thereby permitting diffusion of solvated active compound within the matrix. Diffusion coefficients of active compounds in hydrogels are characteristically high, and for highly water-swollen gels, the diffusion coefficient of the active compound in the gel may approach the value in pure water. This high diffusion coefficient permits practical release rates from relatively large devices (i.e., it is not necessary to form microparticles). Although hydrogel devices can be prepared, loaded with an active component, stored, dispensed and dosed in the fully hydrated state, it is preferred that they be stored, dispensed, and dosed in a dry state. In addition to stability and convenience, dry state dosing of hydrogel devices can provide good active component release kinetics due to Case II transport (i.e., combination of swelling of hydrogel and diffusion of active compound out through the swollen hydrogel). Preferred materials for forming hydrogels include hydrophilic vinyl and acrylic polymers, polysaccharides such as calcium alginate, and poly(ethylene oxide). Especially preferred are poly(2-hydroxyethyl methacrylate), poly(acrylic acid), poly(methacrylic acid), poly(N-vinyl-2-pyrolidinone), poly(vinyl alcohol) and their copolymers with each other and with hydrophobic monomers such as methyl methacrylate, vinyl acetate, and the like. Also preferred are hydrophilic polyurethanes containing large poly(ethylene oxide) blocks. Other preferred materials include hydrogels comprising interpenetrating networks of polymers, which may be formed by addition or by condensation polymerization, the components of which may comprise hydrophilic and hydrophobic monomers such as those just enumerated.

Non-eroding matrix tablets can be made by tabletting methods common in the pharmaceutical industry. Preferred embodiments of non-eroding matrix tablets contain about 1 to about 80% active component, about 5 to about 50% insoluble matrix materials such as cellulose, cellulose acetate, or ethylcellulose, and optionally about 5 to about 85% plasticizers, pore formers or solubilizing excipients, and optionally about 0.25 to about 2% of a tabletting lubricant, such as magnesium stearate, sodium stearyl fumarate, zinc stearate, calcium stearate, stearic acid, polyethyleneglycol-8000, talc, or mixtures of magnesium stearate with sodium lauryl sulfate. These materials can be blended, granulated, and tabletted using a variety of equipment common to the pharmaceutical industry.

A non-eroding matrix multiparticulate comprises a plurality of active component-containing particles, each particle comprising a mixture of active component with one or more excipients selected to form a matrix capable of limiting the dissolution rate of the active component into an aqueous medium. The matrix materials useful for this embodiment are generally water-insoluble materials such as triglycerides, waxes, cellulose, or other water-insoluble polymers. If needed, the matrix materials may optionally be formulated with water-soluble materials that can be used as binders or as permeability-modifying agents. Matrix materials useful for the manufacture of these dosage forms include microcrystalline cellulose such as Avicel® (FMC Corp., Philadelphia, Pa.), including grades of microcrystalline cellulose to which binders such as hydroxypropyl methyl cellulose have been added, waxes such as paraffin, modified vegetable oils, camauba wax, hydrogenated castor oil, beeswax, and the like, as well as synthetic polymers such as poly(vinyl chloride), poly(vinyl acetate), copolymers of vinyl acetate and ethylene, polystyrene, and the like. Water-soluble release modifying agents that can optionally be formulated into the matrix include water-soluble polymers such as HPC, HPMC, methyl cellulose, PVP, PEO, PVA, xanthan gurn, carrageenan, and other such natural and synthetic materials. In addition, materials that function as release-modifying agents include water-soluble materials such as sugars or salts. Preferred water-soluble materials include lactose, sucrose, glucose, and mannitol, as well as HPC, HPMC, and PVP. In addition, any of the solubilizing acids or excipients previously mentioned can be incorporated into matrix multiparticulates to increase the release rate of the active component, increase the total quantity of the active component released, and potentially increase absorption and consequently the bioavailability of the active component, particularly from matrix formulations that release the active component over a period of six hours or longer.

A preferred process for manufacturing matrix multiparticulates is the extrusion/spheronization process. For this process, the active component is wet-massed with a binder, extruded through a perforated plate or die, and placed on a rotating disk. The extrudate ideally breaks into pieces, which are rounded into spheres, spheroids, or rounded rods on the rotating plate. A preferred process and composition for this method involves using water to wet-mass a blend comprising about 20 to about 99% of microcrystalline cellulose blended with, correspondingly, about 80 to about 1% active component.

A preferred process for manufacturing matrix multiparticulates is the rotary granulation process. For this process the active component and excipients such as microcrystalline cellulose are placed in a rotor bowl in a fluid-bed processor. The active compound and excipient are fluidized, while spraying a solution that binds the active compound and excipients together in granules or multiparticulates. The solution sprayed into the fluid bed can be water or aqueous solutions or suspensions of binding agents such as polyvinylpyrrolidone or hydroxypropylmethylcellulose. A preferred composition for this method can comprise about 1 to about 80% active component, about 10 to about 60% microcrystalline cellulose, and about 0 to about 25% binding agent.

A further preferred process for manufacturing matrix multiparticulates involves coating the active component, matrix-forming excipients, and if desired, release-modifying or solubilizing excipients onto seed cores such as sugar seed cores known as non-pareils. Such coatings can be applied by many methods known in the pharmaceutical industry, such as spray-coating in a fluid bed coater, spray-drying, and granulation methods such as fluid bed or rotary granulation. Coatings can be applied from aqueous, organic or melt solutions or suspensions.

A further preferred process for manufacturing matrix multiparticulates is the preparation of wax granules via a melt-congeal process. In this process, a desired amount of the active component is stirred with liquid wax to form a homogeneous mixture, cooled and then forced through a screen to form granules. Alternatively, the homogeneous mixture can be fed to a spinning disc where the mixture is broken up into droplets as it is spun off the edges of the disc. These droplets are then cooled, and solidify before landing in a collection chamber. Preferred matrix materials are waxy substances. Especially preferred are hydrogenated castor oil, glyceryl behenate, microcrystalline wax, camauba wax, and stearyl alcohol.

A further preferred process for manufacturing matrix multiparticulates involves using an organic solvent to aid mixing of the active component with the matrix material. This technique can be used when it is desired to utilize a matrix material with an unsuitably high melting point that, if the material were employed in a molten state, would cause decomposition of the active compound or of the matrix material, or would result in an unacceptable melt viscosity, thereby preventing mixing of the active component with the matrix material. Active component and matrix material may be combined with a modest amount of solvent to form a paste, and then forced through a screen to form granules from which the solvent is then removed. Alternatively, the active component and matrix material may be combined with enough solvent to completely dissolve the matrix material and the resulting solution (which may contain solid active compound particles) spray dried to form the particulate dosage form. This technique is preferred when the matrix material is a high molecular weight synthetic polymer such as a cellulose ether or cellulose ester. Solvents typically employed for the process include acetone, ethanol, isopropanol, ethyl acetate, and mixtures of two or more.

A further process for manufacturing matrix multiparticulates involves using an aqueous solution or suspension of the active component and matrix forming materials. The solution or suspension can be spray dried or sprayed or dripped into a quench bath or through a light chamber to initiate crosslinking of matrix materials and solidify the droplets. In this manner matrices can be made from latexes (e.g., dispersed ethyl cellulose with a plasticizer such as oleic acid or with a volatile water miscible solvent such as acetone or ethanol) by spray-drying techniques. Matrices can also be made in this manner by crosslinking a water soluble polymer or gum. For example, sodium alginate can be crosslinked by spraying into a solution containing soluble calcium salts, polyvinyl alcohol can be crosslinked by spraying into a solution containing gluteraldehyde, and di- and tri-acrylates can be crosslinked by UV irradiation.

Once formed the active component matrix multiparticulates may be blended with compressible excipients such as lactose, mannitol, microcrystalline cellulose, dicalcium phosphate, and the like and the blend compressed to form a tablet. Disintegrants such as sodium starch glycolate, sodium croscarmellose, or crosslinked poly(vinyl pyrrolidone) are also usefully employed. Tablets prepared by this method disintegrate when placed in an aqueous medium (such as the GI tract), thereby exposing the multiparticulate matrix, which releases the active component. Active component matrix multiparticulates may also be filled into capsules, such as hard gelatin capsules. Multiparticulates can also be directly dosed as a sachet that is mixed with water or other suitable drink, or can be sprinkled directly on food.

A further embodiment of a matrix system has the form of a hydrophilic matrix tablet containing an active component that eventually dissolves or disperses in water and an amount of hydrophilic polymer sufficient to provide a useful degree of control over the release of the active component. Active component can be released from such matrices by diffusion, erosion or dissolution of the matrix, or a combination of these mechanisms. Hydrophilic polymers useful for forming a hydrophilic matrix include HPMC, HPC, hydroxy ethyl cellulose (HEC), PEO, PVA, polyacrylic acid, xanthan gum, carbomer, carrageenan, and zooglan. A preferred material is HPMC. Other similar hydrophilic polymers may also be employed. In use, the hydrophilic material is swollen by, and eventually dissolves or disperses in, water. The active component release rate from hydrophilic matrix formulations may be controlled by the amount and molecular weight of hydrophilic polymer employed. In general, using a greater amount of the hydrophilic polymer decreases the release rate, as does using a higher molecular weight polyrmer. Using a lower molecular weight polymer increases the release rate. The release rate may also be controlled by the use of water-soluble additives such as sugars, salts, or soluble polymers. Examples of these additives are sugars such as lactose, sucrose, or mannitol, salts such as NaCl, KCl, NaHCO₃, and water-soluble polymers such as PVP, low molecular weight HPC or HMPC or methyl cellulose. In general, increasing the fraction of soluble material in the formulation increases the release rate. In addition, any of the solubilizing acid excipients previously mentioned can be incorporated into matrix tablets to increase the release rate of active component, increase the total quantity of active component released, and potentially increase absorption and consequently the bioavailability of active component, particularly from matrix formulations that release active component over a period of six hours or longer. A hydrophilic matrix tablet typically comprises about 1 to about 90% by weight of the active component and about 80 to about 10% by weight of polymer.

A preferred hydrophilic matrix tablet comprises, by weight, about 3% to about 80% active component, about 5% to about 35% HPMC, about 0% to about 55% lactose or mannitol, about 0% to about 15% PVP, about 0% to about 20% microcrystalline cellulose, and about 0.25% to about 2% magnesium stearate.

Mixtures of polymers and/or gums can also be utilized to make hydrophilic matrix systems. For example, homopolysaccharide gums such as galactomannans (e.g., locust bean gum or guar gum) mixed with heteropolysaccharide gums (e.g., xanthan gum or its derivatives) can provide a synergistic effect that in operation provides faster forming and more rigid matrices for the release of active compound (See, for example, U.S. Pat. Nos. 5,455,046 and 5,512,297). Optionally, crosslinking agents such as calcium salts can be added to improve matrix properties.

Hydrophilic matrix formulations that eventually dissolve or disperse can also be made in the form of multiparticulates. Hydrophilic matrix multiparticulates can be manufactured by the techniques described previously for non-eroding matrix multiparticulates. Preferred methods of manufacture are layering active component, a hydrophilic matrix material, and if desired release modifying agents onto seed cores (e.g., non-pareils) via a spray-coating process or forming multiparticulates by granulation, such as by rotary granulation of active component, hydrophilic matrix material, and if desired, release modifying agents.

The matrix systems as a class often exhibit non-constant release of the active compound from the matrix. This result may be a consequence of the diffusive mechanism of active compound release, and modifications to the geometry of the dosage form and/or coating or partially coating the dosage form can be used to advantage to make the release rate of the active compound more constant as detailed below.

In a further embodiment, an active component matrix tablet is coated with an impermeable coating, and an orifice (for example, a circular hole or a rectangular opening) is provided by which the content of the tablet is exposed to the aqueous GI tract. These embodiments are along the lines of those presented in U.S. Pat. No. 4,792,448 to Ranade, and as described by Hansson et al., J. Pharm. Sci., 77 (1988) 322-324. The opening is typically of a size such that the area of the exposed underlying active component constitutes less than about 40% of the surface area of the device, preferably less than about 15%.

In another embodiment, an active component matrix tablet is coated with an impermeable material on part of its surface, e.g., on one or both tablet faces, or on the tablet radial surface.

In another embodiment, an active component matrix tablet is coated with an impermeable material and an opening for active compound transport produced by drilling a hole through the coating. The hole may be through the coating only, or may extend as a passageway into the tablet.

In another embodiment, an active component matrix tablet is coated with an impermeable material and a passageway for active compound transport produced by drilling a passageway through the entire tablet.

In another embodiment, an active component matrix tablet is coated with an impermeable material and one or more passageways for active compound transport are produced by removing one or more strips from the impermeable coating or by cutting one or more slits through the coating, preferably on the radial surface or land of the tablet.

In another embodiment, an active component matrix tablet is shaped in the form of a cone and completely coated with an impermeable material. A passageway for active compound transport is produced by cutting off the tip of the cone.

In another embodiment, an active component matrix tablet is shaped in the form of a hemisphere and completely coated with an impermeable material. A passageway for active compound transport is produced by drilling a hole in the center of the flat face of the hemisphere.

In another embodiment, an active component matrix tablet is shaped in the form of a half-cylinder and completely coated with an impermeable material. A passageway for active component transport is produced by cutting a slit through (or removing a strip from) the impermeable coating along the axis of the half-cylinder along the centerline of the flat face of the half-cylinder. Those skilled in the art will appreciate that the geometric modifications to the embodiments described above can be equivalently produced by more than one method.

By “impermeable material” is meant a material having sufficient thickness and impermeability to active component such that the majority of active component is released through the passageway rather than through the “impermeable material” during the time scale of the intended active compound release. Such a coating can be obtained by selecting a coating material with a sufficiently low diffusion coefficient for active component and applying it sufficiently thickly. Materials for forming the impermeable coating of these embodiments include substantially all materials in which the diffusion coefficient of the active component is less than about 10⁻⁷ cm²/sec. It is noted that the preceding diffusion coefficient can be amply sufficient to allow release of active component from a matrix device, as discussed above. However, for a device of the type now under discussion that has been provided with a macroscopic opening or passageway, a material with this diffusion coefficient is effectively impermeable to active component relative to active component transport through the passageway. Preferred coating materials include film-forming polymers and waxes. Especially preferred are thermoplastic polymers, such as poly(ethylene-co-vinyl acetate), poly(vinyl chloride), ethylcellulose, and cellulose acetate. These materials exhibit the desired low permeation rate of active component when applied as coatings of thickness greater than about 100 μm.

A second class of active component sustained-release dosage forms of the present invention includes membrane-moderated or reservoir systems such as membrane-coated diffusion-based capsule, tablet, or multiparticulate. Capsules, tablets and mutiparticulates can all be reservoir systems, such as membrane-coated diffusion-based. In this class, a reservoir of active component is surrounded by a rate-limiting membrane. The active component traverses the membrane by mass transport mechanisms well known in the art, including but not limited to dissolution in the membrane followed by diffusion across the membrane or diffusion through liquid-filled pores within the membrane. These individual reservoir system dosage forms may be large, as in the case of a tablet containing a single large reservoir, or multiparticulate, as in the case of a capsule containing a plurality of reservoir particles, each individually coated with a membrane. The coating can be non-porous, yet permeable to active component (for example, active component may diffuse directly through the membrane), or it may be porous.

Sustained release coatings as known in the art may be employed to fabricate the membrane, especially polymer coatings, such as a cellulose ester or ether, an acrylic polymer, or a mixture of polymers. Preferred materials include ethyl cellulose, cellulose acetate and cellulose acetate butyrate. The polymer may be applied as a solution in an organic solvent or as an aqueous dispersion or latex. The coating operation may be conducted in standard equipment such as a fluid bed coater, a Wurster coater, or a rotary bed coater.

If desired, the permeability of the coating may be adjusted by blending of two or more materials. A particularly useful process for tailoring the porosity of the coating comprises adding a pre-determined amount of a finely-divided water-soluble material, such as sugars or salts or water-soluble polymers to a solution or dispersion (e.g., an aqueous latex) of the membrane-forming polymer to be used. When the dosage form is ingested into the aqueous medium of the GI tract, these water-soluble membrane additives are leached out of the membrane, leaving pores that facilitate release of the active compound. The membrane coating can also be modified by the addition of plastidzers, as known in the art.

A particularly useful variation of the process for applying a membrane coating comprises dissolving the coating polymer in a mixture of solvents chosen such that as the coating dries, a phase inversion takes place in the applied coating solution, resulting in a membrane with a porous structure. Numerous examples of this type of coating system are given in U.S. Pat. No. 5,612,059.

The morphology of the membrane is not of critical importance so long as the permeability characteristics enumerated herein are met. However, specific membrane designs will have membrane morphology constraints in order to achieve the desired permeability. The membrane can be amorphous or crystalline. It can have any category of morphology produced by any particular process and can be, for example, an interfacially-polymerized membrane (which comprises a thin rate-limiting skin on a porous support), a porous hydrophilic membrane, a porous hydrophobic membrane, a hydrogel membrane, an ionic membrane, and other such membrane designs which are characterized by controlled permeability to active component.

A useful reservoir system embodiment is a capsule having a shell comprising the material of the rate-limiting membrane, including any of the membrane materials previously discussed, and filled with an active component composition. A particular advantage of this configuration is that the capsule may be prepared independently of the active compound composition, thus process conditions that would adversely affect the active compound can be used to prepare the capsule. A preferred embodiment is a capsule having a shell made of a porous or a permeable polymer made by a thermal forming process. An especially preferred embodiment is a capsule shell in the form of an asymmetric membrane; i.e., a membrane that has a thin dense region on one surface and most of whose thickness is constituted of a highly permeable porous material. A preferred process for preparation of asymmetric membrane capsules comprises a solvent exchange phase inversion, wherein a solution of polymer, coated on a capsule-shaped mold, is induced to phase-separate by exchanging the solvent with a miscible non-solvent. Examples of asymmetric membranes useful in this invention are disclosed in U.S. Pat. Nos. 5,698,220 and 5,612,059.

Tablets can also be reservoir systems. Tablet cores containing active component can be made by a variety of techniques standard in the pharmaceutical industry. These cores can be coated with a rate-controlling coating as described above, which allows the active component in the reservoir (tablet core) to diffuse out through the coating at the desired rate.

Another embodiment of reservoir systems comprises a multiparticulate wherein each particle is coated with a polymer designed to yield sustained release of active component. The multiparticulate particles each comprise active component and one or more excipients as needed fcor fabrication and performance. The size of individual particles, as previously mentioned, is generally between about 50 μm and about 3 mm, although beads of a size outside this range may also be useful. In general, the beads comprise active component and one or more binders. As it is generally desirable to produce dosage forms that are small and easy to swallow, beads that contain a high fraction of active component relative to excipients are preferred. Binders useful in fabrication of these beads include microcrystalline cellulose (e.g., Avicel®, FMC Corp.), HPC, HPMC, and related materials or combinations thereof. In general, binders that are useful in granulation and tabletting, such as starch, pregelatinized starch, and PVP may also be used to form multiparticulates.

Reservoir system active component multiparticulates may be prepared using techniques known to those skilled in the art, including, but not limited to, the techniques of extrusion and spheronization, wet granulation, fluid bed granulation, melt-congealing, and rotary bed granulation. In addition, the beads may also be prepared by building the active component composition (active component plus excipients) up on a seed core (such as a non-pareil seed) by an active compound-layering technique such as powder coating or by applying the active component composition by spraying a solution or dispersion of active component in an appropriate binder solution onto seed cores in a fluidized bed such as a Wurster coater or a rotary processor. An example of a suitable composition and method is to spray a dispersion of an active component/thydroxypropylcellulose composition in water.

A preferred method for manufacturing the multiparticulate cores of this embodiment is the extrusion/spheronization process, as previously discussed for matrix multiparticulates. A preferred process and composition for this method involves using water to wet-mass a blend of about 5 to about 99% of microcrystalline cellulose with correspondingly about 95 to about 1% active component. Especially preferred is the use of about 95 to about 50% microcrystalline cellulose with correspondingly about 5 to about 50% active component.

A preferred process for making multiparticulate cores of this embodiment is the rotary-granulation process, as previously discussed for matrix multiparticulates. Another preferred process for making multiparticulate cores of this embodiment is the melt-congeal process, as previously discussed for matrix multiparticulates. Another preferred process for making multiparticulate cores of this embodiment is the process of coating seed cores with active component and optionally other excipients, as previously discussed for matrix multiparticulates.

A sustained release coating as is known in the art, especially polymer coatings, may be employed to fabricate the membrane, as previously discussed for reservoir systems. Suitable and preferred polymer coating materials, equipment, and coating methods also include those previously discussed.

The rate of active component release from the coated multiparticulates can also be controlled by factors such as the composition and binder content of the active compound-containing core, the thickness and permeability of the coating, and the surface-to-volume ratio of the multiparticulates. It will be appreciated by those skilled in the art that increasing the thickness of the coating will decrease the release rate, whereas increasing the permeability of the coating or the surface-to-volume ratio of the multiparticulates will increase the release rate. If desired, the permeability of the coating may be adjusted by blending of two or more materials. A useful series of coatings comprises mixtures of water-insoluble and water-soluble polymers, for example, ethylcellulose and hydroxypropyl methylcellulose, respectively. A particularly useful modification to the coating is the addition of finely divided water-soluble material, such as sugars or salts. When placed in an aqueous medium, these water-soluble membrane additives are leached out of the membrane, leaving pores that facilitate delivery of the active compound. The membrane coating may also be modified by the addition of plasticizers, as is known to those skilled in the art. A particularly useful variation of the membrane coating utilizes a mixture of solvents chosen such that as the coating dries, a phase inversion takes place in the applied coating solution, resulting in a membrane with a porous structure.

A preferred embodiment is a multiparticulate with cores comprising about 1 to about 50% active component and about 10 to about 70% of one or more of the following: microcrystalline cellulose, lactose, mannitol, glyceryl behenate, stearyl alcohol, microcrystalline wax, PVP, HPC and HPMC. The individual cores are coated with either an aqueous dispersion of ethyl cellulose, which dries to form a continuous film, or a film of cellulose acetate containing PEG, sorbitol or glycerol as a release-modifying agent.

A third class of active component sustained-release dosage forms includes the osmotic delivery devices or “osmotic pumps” as they are known in the art. Osmotic pumps comprise a core containing an osmotically effective composition surrounded by a semipermeable membrane. The term “semipermeable” in this context means that water can pass through the membrane, but solutes dissolved in the core permeate through the membrane at a rate significantly slower than water. In use, when placed in an aqueous environment, the device imbibes water due to the osmotic activity of the core composition. Owing to the semipermeable nature of the surrounding membrane, the contents of the device (including the active compound and any excipients) cannot pass through the non-porous regions of the membrane and are driven by osmotic pressure to leave the device through an opening or passageway pre-manufactured into the dosage form or, alternatively, formed in situ in the GI tract as by the bursting of intentionally-incorporated weak points in the coating under the influence of osmotic pressure, or alternatively, formed in situ in the GI tract by dissolution and removal of water-soluble porosigens incorporated in the coating. The osmotically effective composition includes water-soluble species that generate a colloidal osmotic pressure, and water-swellable polymers. The active component itself (if highly water-soluble) may be an osmotically effective component of the mixture. The active compound composition may be separated from the osmotically effective components by a movable partition or piston.

Materials useful for forming the semipermeable membrane include polyamides, polyesters, and cellulose derivatives. Preferred are cellulose ethers and esters. Especially preferred are cellulose acetate, cellulose acetate butyrate, and ethyl cellulose. Especially useful materials include those that spontaneously form one or more exit passageways, either during manufacturing or when placed in an environment of use. These preferred materials comprise porous polymers, the pores of which are formed by phase inversion during manufacturing, as described below, or by dissolution of a water-soluble component present in the membrane.

A class of materials that have particular utility for forming semipermeable membranes for use in osmotic delivery devices is that of porous hydrophobic polymers or vapor-permeable films, as disclosed by U.S. Pat. No. 5,827,538. These materials are highly permeable to water, but highly impermeable to solutes dissolved in water. These materials owe their high water permeability to the presence of numerous microscopic pores (i.e., pores that are much larger than molecular dimensions). Despite their porosity, these materials are impermeable to molecules in aqueous solution because liquid water does not wet the pores. Water in the vapor phase is easily able to pass across membranes made from these materials. Such membranes are also known as vapor-permeable membranes.

A preferred embodiment of this class of osmotic delivery devices consists of a coated bi-layer tablet. The coating of such a tablet comprises a membrane permeable to water but substantially impermeable to active component and excipients contained within. The coating contains one or more exit passageways in communication with the active component-containing layer for delivering the active component. The tablet core consists of two layers: one layer containing the active component composition (including optional osmotic agents and hydrophilic water-soluble polymers) and another layer consisting of a water-swellable material, with or without additional osmotic agents.

When placed in an aqueous medium, the tablet imbibes water through the membrane, causing the active component composition to form a dispensible aqueous composition, and causing the swellable layer to expand and push against the active component composition, forcing the active component composition out of the exit passageway. The active component composition can swell aiding in forcing the active component out the passageway. Active component can be delivered from this type of delivery system either dissolved or dispersed in the composition forced out of the exit passageway.

Exemplary materials that are useful to form the active component composition, in addition to the active component itself, include HPMC, PEO, and PVP, and other pharmaceutically-acceptable carriers. In addition, osmotic agents such as sugars or salts, especially sucrose, lactose, mannitol, or sodium bitartrate, may be added. Materials that are useful for forming the swelling layer include sodium carboxymethyl cellulose, poly(ethylene oxide), poly(acrylic acid), sodium (poly-acrylate), hydroxypropylmethylcellulose (HPMC), hydroxypropylcellulose (HPC), and other high molecular-weight hydrophilic materials. In addition, osmagents such as sugars or salts may be added. Particularly useful are poly(ethylene oxide)s having a molecular weight from about 5,000,000 to about 7,500,000.

Materials that are useful for forming the coating are cellulose esters, cellulose ethers, and cellulose ester-ethers. Preferred are cellulose acetate and ethylcellulose and optionally with PEG included as permeability modifying component.

The exit passageway must be located on the side of the tablet containing the active component composition. There may be more than one such exit passageway. The exit passageway may be produced by mechanical means or by laser drilling, or by creating a difficult-to-coat region on the tablet by use of special tooling during tablet compression or by other means.

Osmotic systems can also be made with a homogeneous core surrounded by a semipermeable membrane coating. Active component can be incorporated into a tablet core that also contains other excipients that provide sufficient osmotic driving force and optionally solubilizing excipients such as acids. A semipermeable membrane coating can be applied via conventional tablet-coating techniques such as using a pan coater. An active compound-delivery passageway can then be formed in this coating by drilling a hole in the coating, either by use of a laser or other mechanical means. Alternatively, the passageway may be formed by rupturing a portion of the coating or by creating a region on the tablet that is difficult to coat, as described above.

The core can consist of one or more pharmaceutically active compounds, water-soluble compounds for inducing osmosis, non-swelling solubilizing agents, non-swelling (water-soluble or water-insoluble) wicking agents, swellable hydrophilic polymers, binders and lubricants.

The osmotically active (water-soluble) agent is typically a sugar alcohol such as mannitol or sorbitol, or sugars in combination with polysaccharides such as dextrose and maltose, or a physiologically tolerable ionic salt that is compatible with the other components such as sodium or potassium chloride. Another osmotic agent is urea. Examples of water-soluble compounds for inducing osmosis are: inorganic salts such as magnesium chloride or magnesium sulfate, lithium, sodium or potassium chloride, lithium, sodium or potassium hydrogen or dihydrogen phosphate, salts of organic acids such as sodium or potassium acetate, magnesium succinate, sodium benzoate, sodium citrate or sodium ascorbate; carbohydrates such as sorbitol or mannitol (hexite), arabinose, dextrose, ribose or xylose (pentosene), glucose, fructose, galactose or mannose (hexosene), sucrose, maltose or lactose (disaccharides) or raffinose (trisaccharides); water-soluble amino acids such as glycine, leucine, alanine or methionine, urea and the like, and mixtures thereof. These water-soluble excipients may be present in the core in amounts by weight of about 0.01 to about 45%, based on the total weight of the therapeutic system.

Non-swelling solubilizing agents include (a) agents that inhibit crystal formation of the active agent or otherwise act by complexation therewith; (b) high HLB (hydrophilic-lipophilic balance) micelle-forming surfactants, particularly non-ionic and/or anionic surfactants: (c) citrate esters; and combinations thereof, particularly combinations of complexing agents and anionic surfactants. Examples of agents that inhibit crystal formation of the active agent or otherwise acts by complexation therewith include polyvinylpyrrolidone, polyethyleneglycol (particularly PEG 8000), cyclodextrins and modified cyclodextrins. Examples of high HLB, micelle forming surfactants include Tween 20, Tween 60, Tween 80, polyoxyethylene or polyethylene-containing surfactants, or other long chain anionic surfactants, particularly sodium lauryl sulfate. Examples of citrate ester derivatives that are preferred are the alkyl esters, particularly triethyl citrate. Combinations of these that are particularly preferred are polyvinylpyrrolidone with sodium lauryl sulfate and polyethyleneglycol with sodium lauryl sulfate.

Non-swelling wicking (wetting) agents are used to create channels or pores in the core of the tablet. This facilitates channeling of water through the core by physisorption. Preferred wicking agents do not swell to any appreciable degree. These materials can be water soluble or water insoluble materials. Water-soluble materials suitable for acting as wicking (wetting) agents include surface-active compounds, i.e., surfactants, e.g., anionic surfactants of the alkylsulfate type such as sodium, potassium or magnesium lauryl sulfate, n-tetradecylsulfate, n-hexadecyl sulfate or n-octadecylsulfate; of the alkyl ether sulfate type, e.g., sodium, potassium or magnesium n-dodecyloxyethyl sulfate, n-tetradecyloxyethyl sulfate, n-hexadecyloxyethyl sulfate or n-octadecyloxyethyl sulfate; or of the alkylsulfonate type, e.g. sodium potassium or magnesium n-dodecanesulfonate, n-tetradecanesulfonate, n-hexadecanesulfonate or n-octadecanesulfonate. Further suitable surfactants are nonionic surfactants of the fatty acid polyhydoxy alcohol ester type such as sorbitan monolaurate, sorbitan tristerate or triolate, polyethylene glycol fatty acid ester such as polyoxyethyl stearate, polyethylene glycol 400 stearate, polyethylene glycol 2000 stearate, preferably polyethylene oxide/propylene oxide block copolymers of the Pluronic® (BASF, Parsippany, N.J.) or Synperonic® (ICI Surfactants, Everberg, Belgium) type, polyglycerol-fatty acid esters or glyceryl-fatty acid esters. Especially suitable is sodium lauryl sulfate. When present, these surfactants should be preferable present from about 0.2 to about 2% based on the total core weight. Other soluble wicking (wetting) agents include low molecular weight polyvinyl pyrrolidone and m-pyrol.

Insoluble materials suitable for acting as wicking (wetting) agents include, but are not limited to, colloidal silicon dioxide, kaolin, titanium dioxide, fumed silicon dioxide, alumina, niacinamide, bentonite, magnesium aluminum silicate, polyester, polyethylene. Particularly suitable insoluble wicking agents include colloidal silicon dioxide.

Suitable wall materials for forming the semi-permeable wall include microporous materials described in U.S. Pat. Nos. 3,916,899 and 3,977,404. It is possible to use acylated cellulose derivatives (cellulose esters) which are substituted by one to three acetyl groups or by one or two acetyl groups and a further acyl other than acetyl, e.g., cellulose acetate, cellulose triacetate, agar acetate, amylose acetate, beta glucan acetate, beta glucan triacetate, ethyl cellulose, cellulose acetate ethyl carbamate, cellulose acetate phthalate, cellulose acetate methyl carbamate, cellulose acetate succinate, cellulose acetate dimethylaminoaceate, cellulose acetate ethyl carbonate, cellulose acetate chloroacetate, cellulose acetate ethyl oxalate, cellulose acetate methylsulfonate, cellulose acetate butyl sulfonate, cellulose acetate propionate, cellulose acetate octate, cellulose acetate laurate, cellulose acetate p-toluenesulfonate, cellulose acetate butyrate, and other cellulose acetate derivatives. Suitable semi-permeable membrane materials are also triacetate of locust bean gum, methyl cellulose, hydroxypropyl methylcellulose and polymeric epoxides, copolymers of alkylene oxides, poly(vinyl methyl) ether polymers and alkyl glycidyl ethers, polyglycols or polylactic acid derivatives and further derivatives thereof. It is also possible to use mixtures of insoluble polymers, which when coated form a semi-permeable film, e.g. water insoluble acrylates, e.g., the copolymer of ethyl acrylate and methyl methacrylate.

A second, water-soluble component can be added to increase the permeability of the coating. Preferred water-soluble components are C₂-C₄ alkylene glycol, preferably polyethylene glycol.

An embodiment of active component sustained release osmotic dosage forms of this invention comprises an osmotic active component-containing tablet, which is surrounded by an asymmetric membrane, where said asymmetric membrane possesses one or more thin dense regions in addition to less dense porous regions. This type of membrane, similar to those used in the reverse-osmosis industry, generally allows higher osmotic fluxes of water than can be obtained with a dense membrane. When applied to a active compound formulation, e.g., a tablet, an asymmetric membrane allows high active compound fluxes and well-controlled sustained active compound release. This asymmetric membrane comprises a semipermeable polymeric material, that is, a material which is permeable to water, and substantially impermeable to salts and the active component.

Materials useful for forming the semipermeable membrane include polyamides, polyesters, and cellulose derivatives. Preferred are cellulose ethers and esters. Especially preferred are cellulose acetate, cellulose acetate butyrate, and ethyl cellulose. Especially useful materials include those which spontaneously form one or more exit passageways, either during manufacturing or when placed in an environment of use. These preferred materials comprise porous polymers, the pores of which are formed by phase inversion during manufacturing, as described above, or by dissolution of a water-soluble component present in the membrane.

The asymmetric membrane is formed by a phase-inversion process. The coating polymer, e.g., ethylcellulose or cellulose acetate, is dissolved in a mixed solvent system comprising a mixture of solvents (e.g., acetone) and non-solvents (e.g., water) for the ethylcellulose or cellulose acetate. The components of the mixed solvent are chosen such that the solvent (e.g., acetone) is more volatile than the non-solvent (e.g., water). When a tablet is dipped into such a solution, removed and dried, the solvent component of the solvent mixture evaporates more quickly than the non-solvent. This change in solvent composition during drying causes a phase-inversion, resulting in precipitation of the polymer on the tablet as a porous solid with a thin dense outer region. This outer region possesses multiple pores through which active compound delivery can occur.

In a preferred embodiment of an asymmetric membrane-coated tablet, the polymer/solvent/non-solvent mixture is sprayed onto a bed of tablets in a tablet-coating apparatus such as a Freund HCT-30 tablet coater (Freund Industrial Co., Tokyo, Japan).

In the environment of use, e.g., the GI tract, water is imbibed through the semipermeable asymmetric membrane into the tablet core. As soluble material in the tablet core dissolves, an osmotic pressure gradient across the membrane builds. When the hydrostatic pressure within the membrane enclosed core exceeds the pressure of the environment of use (e.g., the GI lumen), the active component containing solution is “pumped” out of the dosage form through preformed pores in the semipermeable membrane. The constant osmotic pressure difference across the membrane results in a constant well-controlled delivery of active component to the use environment. A portion of the active component dissolved in the tablet also exits via diffusion.

In this asymmetric-membrane-coated tablet embodiment, high solubility salts of the active component are preferred. Also preferred are the inclusion of one or more solubilizing excipients, ascorbic acid, erythorbic acid, citric acid, fumaric acid, succinic acid, tartaric acid, sodium bitartrate, glutamic acid, aspartic acid, partial glycerides, glycerides, glyceride derivatives, polyethylene glycol esters, polypropylene glycol esters, polyhydric alcohol esters, polyoxyethylene ethers, sorbitan esters, polyoxyethylene sorbitan esters, saccharide esters, phospholipids, polyethylene oxide-polypropylene oxide block co-polymers, and polyethylene glycols. Most preferred are solubilizing excipients fumaric acid, ascorbic acid, succinic acid, and aspartic acid.

Osmotic tablets can also be made with a core tablet containing osmogents and/or solubilizing excipients surrounded first by a active compound containing layer and then second a semipermeable coating. The core tablet containing osmotic agents and/or solubilizing excipients can be made by standard tabletting methods known in the pharmaceutical industry. The semipermeable coating can then be applied to the layered core by many processes known in the art such as spray-coating or dip-coating methods described previously in these specifications. The active component-containing layer may be applied around the core by spray-coating methods where a solution or slurry of active compound and excipients is coated onto the tablet core. The active component and excipients may also be layered around the tablet core by making a “layered” type of configuration using a tablet press to form a second active compound-containing layer around the tablet core. This type of compression coating method can be used to apply a powder coating (without solvents) around a tablet core.

Another embodiment of sustained release active component osmotic dosage forms of this invention consists of active component multiparticulates coated with an asymmetric membrane. Active component-containing multiparticulates are prepared by, for example, extrusion/spheronization or fluid bed granulation, or by coating non-pareil seeds with a mixture of active component and a water-soluble polymer, as described above. Active component-containing multiparticulates are then spray-coated with a solution of a polymer in a mixture of a solvent and a non-solvent, as described above, to form asymmetric-membrane-coated multiparticulates. This spray-coating operation is preferably carried out in a fluid bed coating apparatus, e.g., a Glatt GPCG-5 fluid bed coater Glatt Air Techniques, Inc., Ramsey, N.J.). The polymer used for forming the semipermeable asymmetric membrane is chosen as described above for asymmetric-membrane coated tablets. Likewise, excipients for the multiparticulate cores can be chosen as described above for asymmetric-membrane coated tablets.

Osmotic capsules can be made using the same or similar components to those described above for osmotic tablets and multiparticulates. The capsule shell or portion of the capsule shell can be semipermeable and made of materials described above. The capsule can then be filled either by a powder or liquid comprising active component, excipients that provide osmotic potential, and optionally solubilizing excipients. The capsule core can also be made such that it has a bilayer or multilayer composition analogous to the bilayer tablet described above.

A fourth class of active component sustained release dosage forms useful in the methods of this invention comprises coated swellable tablets and multiparticulates, as described in co-pending commonly assigned U.S. application Ser. No. 07/296,464, filed Jan. 12, 1989 (published as EP 378404 A2; Jul. 7, 1990). Coated swellable tablets comprise a tablet core comprising active component and a swelling material, preferably a hydrophilic polymer, coated with a membrane that contains holes or pores through which, in the aqueous use environment, the hydrophilic polymer can extrude and carry out the active component. Alternatively, the membrane may contain polymeric or low molecular weight water soluble porosigens which dissolve in the aqueous use environment, providing pores through which the hydrophilic polymer and the active component may extrude. Examples of porosigens are water-soluble polymers such as hydroxypropylmethylcellulose, and low molecular weight compounds like glycerol, sucrose, glucose, and sodium chloride. In addition, pores may be formed in the coating by drilling holes in the coating using a laser or other mechanical means. In this fourth class of active component sustained release dosage forms, the membrane material may comprise any film-forming polymer, including polymers which are water permeable or impermeable, provided that the membrane deposited on the tablet core is porous or contains water-soluble porosigens or possesses a macroscopic hole for water ingress and active component release. Multiparticulates (or beads) may be similarly prepared, with a active component/swellable material core, coated by a porous or porosigen-containing membrane. Embodiments of this fourth class of active component sustained release dosage forms may also be multilayered, as described in EP 378 404 A2.

Sustained release fomnulations may also be prepared with a portion of the dose released initially rapidly, followed by sustained release of the remaining portion of the dose, thus providing continuous administration.

Formulations that release a portion of the dose as a bolus shortly after administration and then release the remaining portion of the dose at a sustained release rate over time, such as over 2 hours to 18 hours or longer, can be made by a variety of methods. For example, a bilayer tablet can be formed with one layer having a sustained release matrix and the other layer an immediate release composition. Upon ingestion, the immediate release layer disintegrates leaving only the matrix tablet to provide sustained release. In another example, a drug coating can be applied over a matrix or osmotic tablet or over sustained release multiparticulates. The coating can be applied using typical coating equipment standard to the pharmaceutical industry. The active compound can either be a solution or in suspension and is typically mixed with a water soluble polymer in the coating solution. In addition, a combination dosage form can be made by mixing sustained release multiparticulates and immediate release multiparticulates in one dosage form. A preferred method of making a formulation that has an immediate release component and a controlled-release component is to apply a compression coating around an osmotic tablet.

Osmotic tablets comprise a tablet core that contains active compound and may contain excipients that have an osmotic potential greater than the fluid in the environment of use or contain water swellable materials. The tablet cores are surrounded by a semipermeable coating that allows water to be imbibed into the tablet core. In operation it is important that this semipermeable coating remain intact, if the coating is cracked or disrupted dose dumping could occur or the release rate could significantly increase. A compression coating is made by compressing a powder granulation around a tablet core to form a outer layer or coating. This is done in specialized tablet presses where the inner core is place in the powder/granulation during the compression step. Applying an immediate release active compound layer around an osmotic tablet core can be done without cracking or disrupting the semipermeable coating and thus, without affecting the release rate from the osmotic tablet within the compression coating.

The EP₄ receptor selective agonist of formula I or estrogen or estrogen agonist/antagonist or combination thereof can also be administered continuously by infusion. For example, the EP₄ receptor selective agonist of formula I or estrogen or estrogen agonist/antagonist or combination thereof in a pharmaceutically acceptable carrier or diluent can be administered in either a clinical or outpatient setting by infusion pump. Infusion pumps such as the Aim Plus® (Abbott Laboratories, Abbott Park, Ill.); IVAC® 570, 572, 597, 598 or MedSystem III® (Alaris Medical Systems, Inc., San Diego, Calif.), Bard PCA II® or Fluent® (Bard Access Systems, Inc., Salt Lake City, Utah); Baxter Sabretek 6060® (Baxter Healthcare Corp., Deerfield, Ill.)(Graseby 500, 505, 9100, 9200, 9300, 9400 or 9500 (Graseby Medical Ltd., Wafford, Hertfordshire, UK); and Cadd TPN 5700®, Cadd Prizm®, Cadd Plus 5400®, and Cadd PCA 5800® (Sims Deltec, Inc., St. Paul, Minn.) are non-limiting examples of infusion pumps that can be employed for the continuous administration of the EP₄ receptor selective agonist of formula I or estrogen or estrogen agonist/antagonist or combination thereof.

Since the present invention relates to treatment with a combination of two active ingredients that may be administered separately, the invention also relates to combining separate pharmaceutical compositions in kit form. The kit includes two separate pharmaceutical compositions: An EP₄ receptor selective agonist of formula I, such as 5-(3-{2S-[3R-hydroxy-4-(3-trifluoromethyl-phenyl)-butyl}-5-oxo-pyrrolidin-1-yl}-propyl)-thiophene-2-carboxylic acid or a pharmaceutically acceptable salt thereof in a first unit dosage form; and an estrogen, conjugated estrogens or a selective estrogen agonist/antagonist in a second unit dosage form. The kit includes a container for containing the separate compositions such as a divided bottle or a divided foil packet, however, the separate compositions may also be contained within a single, undivided container. Typically the kit includes directions for the administration of the separate components to a mammal for the treatment of musculoskeletal frailty. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It is desirable to provide a memory aid on a card insert, e.g., in the form of numbers next to the tablets or capsules whereby the numbers correspond with the days of the regimen that the tablets or capsules so specified should be ingested. Another example of such a memory aid is a calendar printed on the card e.g., as follows “First Week, Monday, Tuesday, . . . etc. . . . Second Week, Monday, Tuesday, . . .” etc. Other variations of memory aids will be readily apparent. A “daily dose” can be a single tablet or capsule or several pills or capsules to be taken on a given day. For example, a daily dose of an estrogen, conjugated estrogens or an estrogen agonist/antagonist can consist of one tablet or capsule while a daily dose of an EP₄ receptor selective agonist of formula I, such as 5-(3-{2S-[3R-hydroxy-4-(3-trifluoromethyl-phenyl)-butyl-5-oxo-pyrrolid in-1-yl}-propyl)-thiophene-2-carboxylic acid or a pharmaceutically acceptable salt thereof, can consist of several tablets or capsules. The memory aid should reflect this.

In another specific embodiment of the invention a dispenser designed to dispense the daily doses one at a time in the order of their intended use is provided. Preferably, the dispenser is equipped with a memory-aid, so as to further facilitate compliance with the regimen. An example of such a memory-aid is a mechanical counter that indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The compound 5-(3-{2S-[3R-hydroxy-4-(3-trifluoromethyl-phenyl)-butyl]-5-oxo-pyrrolidin-1-yl}-propyl)-thiophene-2-carboxylic acid can be prepared as described in Example 3M of U.S. Patent No. 6,552,067, which procedure is reproduced below.

### EXAMPLE 3M

### 5-(3-{2S-[3R-Hydroxy-4-(3-trifluoromethyl-phenyl)-butyl]-5-oxo-pyrrolidin-1-yl}-propyl)-thiophene-2-carboxylic acid

Step A: 5-(3-{2-Oxo-5R-[3-oxo-4-(3-trifluoromethyl-phenyl)-but-1-enyl]-pyrrolidin-1-yl}-propyl)-thiophene-2-carboxvlic acid methyl ester. Analogous to the procedure described for Example 2A, Step B, the anion derived from [2-oxo-3-(3-trifluoromethyl-phenyl)-propyl]-phosphonic acid dimethyl ester (5.026 g, 17.0 mmol) and NaH (60% by weight in oil, 750 mg, 18.8 mmol) was reacted with 5-[3-(2R-formyl-5-oxo-pyrrolidin-1-yl)-propyl]-thiophene-2-carboxylic acid methyl ester (assumed 18.8 mmol) over 24 h. Purification by medium pressure chromatography (15% acetone in toluene to 20% acetone in toluene) provided 5-(3-{2-oxo-5R-[3-oxo-4-(3-trifluoromethyl-phenyl)-but-1-enyl]-pyrrolidin-1-yl}-propyl)-thiophene-2-carboxylic acid methyl ester (4.02 g). ¹H NMR (CDCl₃) δ7.61 (d, 1H), 7.54 (d, 1H), 7.45 (m, 2H), 7.37 (d, 1H), 6.79 (d, 1H), 6.66 (dd, 1H), 6.20 (d, 1H), 4.16 (m, 1H), 3.90 (s, 2H), 3.84 (s, 3H), 3.60 (m, 1H), 2.89-2.78 (m, 3H), 2.48-2.31 (m, 2H), 2.23 (m, 1H), 1.82 (m, 3H).

Step B: 5-(3-{2R-[3S-Hydroxy-4-(3-trifluoromethyl-phenyl)-but-1-enyl]-5-oxo-pyrrolidin-1-yl}-propyl)-thiophene-2-carboxylic acid methyl ester. Analogous to the procedure described for Example 2A, Step C, 5-(3-{2-oxo-5R-[3-oxo-4-(3-trifluoromethyl-phenyl)-but-1-enyl]-pyrrolidin-1-yl}-propyl)-thiophene-2-carboxylic acid methyl ester (2.63 g, 5.91 mmol) was reduced with catecholborane (1M in THF, 18.8 mL, 18.8 mmol) in the presence of (R)-2-methyl-CBS-oxazaborolidine (1M in toluene, 0.94 mL, 0.94 mmol) at −45° C. over 18 h. The reaction was quenched by addition of 1N HCl and the mixture was stirred for 40 minutes. The organic solution was washed consecutively with ice cold 1N NaOH (3 times), 1N HCl (1 time), water (1 time), and brine. The organic solution was dried (MgSO₄), filtered, and concentrated. Purification by medium pressure chromatography (10% acetone in toluene to 20% acetone in toluene) provided 5-(3-{2R-[3S-hydroxy-4-(3-trifluoromethyl-phenyl)-but-1-enyl]-5-oxo-pyrrolidin-1-yl}-propyl)-thiophene-2-carboxylic acid methyl ester (3 g) as an approximate 4:1 ratio of 3S:3R alcohol diastereomers by ¹H NMR. ¹H NMR (CDCl₃) δ7.60 (d, 1H), 7.50 (d, 1H), 7.41 (m, 3H), 6.79 (d, 1H), 5.70 (dd, 1H), 5.48 (dd, 1H), 4.41 (m, 1H), 4.00 (m, 1H), 3.81 (s, 3H), 3.50 (m, 1H), 2.86-2.77 (m, 5H), 2.42-2.26 (m, 2H), 2.16 (m, 1H), 1.81 (m, 2H), 1.72-1.54 (m, 2H).

Step C: 5-(3-{2S-[3R-Hydroxy-4-(3-trifluoromethyl-phenyl)-butyl]-5-oxo-pyrrolidin-1-yl}-propvl)-thiophene-2-carboxylic acid methyl ester. Analogous to the procedure described for Example 2A, Step D, a mixture of 5-(3-{2R-[3S-hydroxy4-(3-trifluoromethyl-phenyl)-but-1-enyl]-5-oxo-pyrrolidin-1-yl}-propyl)-thiophene-2-carboxylic acid methyl ester (3 g) and 10% palladium on carbon (400 mg) in MeOH (70 mL) was hydrogenated on a Parr shaker at 50 psi for 16 h. Purification by medium pressure chromatography (20% EtOAc in hexanes to 70% EtOAc in hexanes) provided 5-(3-{2S-[3R-hydroxy-4-(3-trifluoromethyl-phenyl)-butyl]-5-oxo-pyrrolidin-1-yl}-propyl)-thiophene-2-carboxylic acid methyl ester (2.26 g). ¹H NMR (CDCl₃) δ7.61 (d, 1H), 7.52-7.38 (m, 4H), 6.81 (d, 1H), 3.83 (m, 4H), 3.63 (m, 2H), 3.00 (m, 1H), 2.85 (m, 3H), 2.74 (m, 1H), 2.34 (m, 2H), 2.10 (m, 1H), 1.98-1.45 (m, 08H).

Step D: 5-(3-{2S-[3R-Hydroxy-4-(3-trifluoromethyl-phenyl)-butyl]-5-oxo-pyrrolidin-1-yl}-propyl)-thiophene-2-carboxylic acid. Analogous to the procedure described for Example 2A, Step E, 5-(3-{2S-[3R-hydroxy-4-(3-trifluoromethyl-phenyl)-butyl]-5-oxo -pyrrolidin-1-yl}-propyl)-thiophene-2-carboxylic acid methyl ester (625 mg) was hydrolyzed with 2N NaOH in MeOH (20 mL) at room temperature over 24 h to provide the title compound of Example 3M (599 mg). ¹H NMR (CDCl₃) δ7.67 (d, 1H), 7.51-7.38 (m, 4H), 6.84 (d, 1H), 3.85 (m, 1H), 3.63 (m, 2H), 3.02 (m, 1H), 2.85 (m, 3H), 2.75 (m, 1H), 2.37 (m, 2H), 2.11 (m, 1H), 2.00-1.45 (m, 8H); MS 470.2 (M+1), 468.2 (M−1).

### EXAMPLE 1

### Continuous Combination Therapy Protocol

### Study Protocol

Prostaglandin E2 (PGE2) restores bone mass by stimulating both bone formation and bone resorption but in favor of bone formation in ovariectomized (OVX) rat skeleton. 5-(3-{2S-[3R-Hydroxy-4-(3-trifluoromethyl-phenyl)-butyl}-5-oxo-pyrrolidin-1-yl}-propyl)-thiophene-2-carboxylic acid, an EP₄ receptor selective agonist, can mimic PGE2's systemic bone anabolic effects when given by daily subcutaneous injection. However, like PGE2, slow release delivery of 5-(3-{2S-[3R-hydroxy-4-(3-trifluoromethyl-phenyl)-butyl}-5-oxo-pyrrolidin-1-yl}-propyl)-thiophene-2-carboxylic acid, by Alzet pump may cause bone loss by stimulating both bone resorption and bone formation but in favor of bone resorption in OVX rat skeleton. Estrogen (17-βestradiol) inhibits bone resorption and turnover, thus preventing bone loss in OVX rats. It was found in this study that combination treatment of 5-(3-{2S-[3R-hydroxy-4-(3-trifluoromethyl-phenyl)butyl}-5-oxo-pyrrolidin-1-yl}-propyl)-thiophene-2-carboxylic acid by slow release and 17-βestradiol (E2) resulted in a more positive bone balance in OVX rats.

Sprague-Dawley (S-D) female rats were sham-operated (n=20) or OVX (n=50) at 3.5 months of age. Three and an half months post-surgery, OVX rats were treated with either vehicle, 5-(3-{2S-[3R-hydroxy-4-(3-trifluoromethyl-phenyl)-butyl}-5-oxo-pyrrolidin-1-yl}-propyl)-thiophene-2-carboxylic acid at 0.3 mg/kg/d (by Alzet pumps in subcutaneous area, 2 ml per hour release rate, for duration of 14 days, replace the pumps at day 15), or 17-βestradiol (E2) at 0.01 mg/kg/d (administered by 30 day release pellets), or combined 5-(3-{2S-[3R-hydroxy-4-(3-trifluoromethyl-phenyl)-butyl}-5-oxo-pyrrolidin-1-yl}-propyl)-thiophene-2-carboxylic acid at 0.3 mg/kg/d (by Alzet pumps in subcutaneous area, 2 ml per hour release rate, for duration of 28 days) and 17-βestradiol (E2) at 0.01 mg/kg/d (administered by 30 day release pellets) for 4 weeks. Total mineral density and cortical bone area of distal femoral metaphysis and femoral shaft were determined by peripheral qualitative computerized tomography (pQCT) as described previously (Ke H. Z. et al., Lasofoxifene protects against the age-related changes in bone mass, bone strength, and total serum cholesterol in intact aged male rats. J. of Bone and Mineral Research, 2001;16:765-773).

### Study Results and Discussion

OVX induced significant decrease in total mineral density (−21%) and cortical bone area (−34%) of distal femoral metaphysis at 3.5 weeks post-surgery as compared to sham controls. Administration of 5-(3-{2S-[3R-hydroxy-4-(3-trifluoromethyl-phenyl)-butyl}-5-oxo-pyrrolidin-1-yl}-propyl)-thiophene-2-carboxylic acid at 0.3 mg/kg/d given by slow release (Alzet pump) caused a further decrease in total mineral density and cortical bone area of distal femoral metaphysis (both at −15%), while 17-βestradiol (E2) pellets given at 0.01 mg/kg/d did not cause significant change as compared with OVX controls. However, total mineral density and cortical bone area of distal femoral metaphysis in OVX rats treated with a combination of 5-(3-{2S-[3R-hydroxy-4-(3-trifluoromethyl-phenyl)-butyl}-5-oxo-pyrrolidin-1-yl}-propyl)-thiophene-2-carboxylic acid and 17-βestradiol (E2) were significantly increased compared with OVX controls (+9% and +25%, respectively). The total mineral density and cortical bone area of distal femoral metaphysis in OVX rats treated with a combination of 5-(3-{2S-[3R-hydroxy-4-(3-trifluoromethyl-phenyl)-butyl}-5-oxo-pyrrolidin-1-yl}-propyl)-thiophene-2-carboxylic acid and 17-βestradiol (E2) did not differ from sham controls, indicating a complete restoration of bone mass to the OVX rat skeleton by combination treatment.

At the femoral shaft, OVX induced no significant change in total mineral density and cortical bone area compared to sham controls. Neither 5-(3-{2S-[3R-hydroxy-4-(3-trifluoromethyl-phenyl)-butyl}-5-oxo-pyrrolidin-1-yl}-propyl)-thiophene-2-carboxylic acid at 0.3 mg/kg/d given by slow release (Alzet pump) nor 17-βestradiol (E2) pellets given at 0.01 mg/kg/d caused significant change in these two parameters. However, total mineral density and cortical bone area of femoral shaft in OVX rats treated with a combination of 5-(3-{2S-[3R-hydroxy-4-(3-trifluoromethyl-phenyl)-butyl}-5-oxo-pyrrolidin-1-yl}-propyl)-thiophene-2-carboxylic acid and 17-βestradiol (E2) were significantly increased compared with OVX controls (+10% and +14%, respectively) and sham controls (+8% and +12%, respectively), indicating that combination treatment of 5-(3-{2S-[3R-hydroxy-4-(3-trifluoromethyl-phenyl)-butyl}-5-oxo-pyrrolidin-1-yl}-propyl)-thiophene-2-carboxylic acid and 17-βestradiol (E2) adds extra bone to the femoral shaft.

These data show that the synergistic effects were found by combination treatment of an EP₄ receptor selective agonist and an estrogen given by continuous slow release administration in OVX rats. These results indicated that combination treatment with an EP₄ receptor selective agonist and an estrogen have more benefits than either alone in postmenopausal bone loss.

All references, patents and patent applications cited herein are hereby incorporated by reference.

What is claimed is:

1. A method of treating a condition which presents with low bone mass in a patient presenting with low bone mass, the method comprising continuously administering to the patient presenting with low bone mass a synergistically effective combination of a first compound and a second compound, the first compound being of the formula I 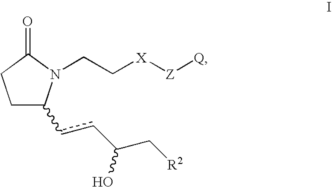
a prodrug thereof, a pharmaceutically acceptable salt of said compound or said prodrug or a stereoisomer or diastereomeric mixture of said compound, prodrug or salt, wherein:
the dotted line is a bond or no bond;
X is —CH₂— or O;
Z is —(CH₂)₃—, thienyl, thiazolyl or phenyl, provided that when X is O, then Z is phenyl;
Q is carboxyl, (C₁-C₄)alkoxylcarbonyl or tetrazolyl;
R² is —Ar or —Ar¹—V—Ar²;
V is a bond, —O—, —OCH₂— or —CH₂O—;
Ar is a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, said partially or fully saturated ring or bicyclic ring optionally having one or two oxo groups substituted on carbon or one or two oxo groups substituted on sulfur; and
Ar¹ and Ar² are each independently a partially saturated, fully saturated or fully unsaturated five to eight membered ring optionally having one to four heteroatoms selected independently from oxygen, sulfur and nitrogen, said partially or fully saturated ring optionally having one or two oxo groups substituted on carbon or one or two oxo groups substituted on sulfur;
said Ar moiety is optionally substituted on carbon or nitrogen, on one ring if the moiety is monocyclic, or on one or both rings if the moiety is bicyclic, with up to three substituents per ring each independently selected from hydroxy, halo, carboxy, (C₁-C₇)alkoxy, (C₁-C₄)alkoxy(C₁-C₄)alkyl, (C₁-C₇)alkyl, (C₂-C₇)alkenyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N-, di-N,N-, di-N,N′- or tri-N,N,N′-(C₁-C₄)alkyl substituted aminocarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl and mono-N- or di-N,N-(C₁-C₄)alkylaminosulfinyl, wherein said alkyl and alkoxy substituents in the definition of Ar are optionally substituted on carbon with up to three fluoro; and
said Ar¹ and Ar² moieties are independently optionally substituted on carbon or nitrogen with up to three substituents each independently selected from hydroxy, halo, carboxy, (C₁-C₇)alkoxy, (C₁-C₄)alkoxy(C₁-C₄)alkyl, (C₁-C₇)alkyl, (C₂-C₇)alkenyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N-, di-N,N-, di-N,N′- or tri-N,N,N′-(C₁-C₄)alkyl substituted aminocarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl and mono-N- or di-N,N-(C₁-C₄)alkylaminosulfinyl, wherein said alkyl and alkoxy substituents in the definition of Ar¹ and Ar² are optionally substituted on carbon with up to three fluoro;
provided that (a) when X is (CH₂)— and Z is —(CH₂)₃—, then R² is not thienyl, phenyl or phenyl monosubstituted with chloro, fluoro, phenyl, methoxy, trifluoromethyl or (C₁-C₄)alkyl; and (b) when X is (CH₂)—, Z is —(CH₂)₃—, and Q is carboxyl or (C₁-C₄)alkoxycarbonyl, then R² is not (i) (C₅-C₇)cycloalkyl or (ii) phenyl, thienyl or furyl each of which may be optionally monosubstituted or disubstituted by one or two substituents selected, independently in the latter case, from halogen atoms, alkyl groups having 1-3 carbon atoms which may be substituted by one or more halogen atoms, and alkoxy groups having 1-4 carbon atoms; and
wherein the second compound is an estrogen, or a pharmaceutically acceptable salt thereof.

2. The method of claim 1 wherein the first compound is of the formula Ia 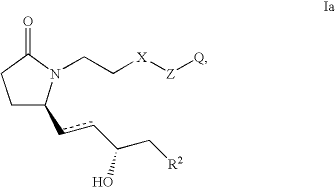
a prodrug thereof, a pharmaceutically acceptable salt of said compound or said prodrug or a stereoisomer or diastereomeric mixture of said compound, prodrug or salt, wherein: 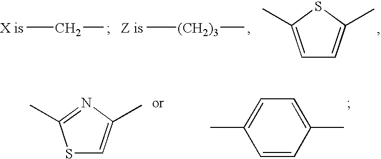
and R² is Ar wherein said Ar moiety is optionally substituted on carbon or nitrogen, on one ring if the moiety is monocyclic, or on one or both rings if the moiety is bicyclic, with up to three substituents per ring each independently selected from hydroxy, halo, carboxy, (C₁-C₇)alkoxy, (C₁-C₄)alkoxy(C₁-C₄)alkyl, (C₁-C₇)alkyl, (C₂-C₇)alkenyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N-, di-N,N-, di-N,N′- or di-N,N,N′-(C₁-C₄)alkyl substituted aminocarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl and mono-N- or di-N,N-(C₁-C₄)alkylaminosulfinyl, wherein said alkyl and alkoxy substituents in the definition of Ar¹ and Ar² are optionally substituted on carbon with up to three fluoro.

3. The method of claim 2 wherein the first compound is of formula Ia, a prodrug thereof, a pharmaceutically acceptable salt of said compound or said prodrug or a stereoisomer or diastereomeric mixture of said compound, prodrug or salt, wherein Ar is cyclohexyl, 1,3-benzodioxolyl, thienyl, naphthyl or phenyl optionally substituted with one or two (C₁-C₄)alkyl, (C₁-C₄)alkoxy, (C₁-C₄)alkoxy(C₁-C₄)alkyl, chloro, fluoro, trifluoromethyl or cyano, wherein said alkyl and alkoxy substituents in the definition of Ar are optionally substituted with up to three fluoro.

4. The method of claim 3 wherein the first compound is of formula Ia, a prodrug thereof, a pharmaceutically acceptable salt of said compound or said prodrug or a stereoisomer or diastereomeric mixture of said compound, prodrug or salt, wherein the dotted line is no bond; Q is carboxy or (C₁-C₄)alkoxylcarbonyl; and Z is

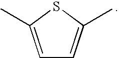

5. The method of claim 4 wherein the first compound is of formula Ia, a prodrug thereof, a pharmaceutically acceptable salt of said compound or said prodrug or a stereoisomer or diastereomeric mixture of said compound, prodrug or salt, wherein Q is carboxy and Ar is phenyl optionally substituted with one (C₁-C₄)alkyl, (C₁-C₄)alkoxy, (C₁-C₄)alkoxy(C₁-C₄)alkyl, chloro, fluoro, trifluoromethyl or cyano, wherein said alkyl and alkoxy substituents in the definition of Ar are optionally substituted with up to three fluoro.

6. The method of claim 5 wherein the first compound is of formula Ia, a prodrug thereof, a pharmaceutically acceptable salt of said compound or said prodrug or a stereoisomer or diastereomeric mixture of said compound, prodrug or salt, wherein Ar is m-trifluoromethylphenyl, m-chlorophenyl or m-trifluoromethoxyphenyl.

7. The method of the claim 6 wherein the first compound is 5-(3-(2S-(3R-hydroxy-4-(3-trifluoromethyl-phenyl)-butyl)-5-oxo-pyrrolidin-1-yl)-propyl)-thiophene-2-carboxylic acid; 5-(3-(2S-(3R-hydroxy-4-(3-trifluoromethoxy-phenyl)-butyl)-5-oxo-pyrrolidin-1-yl)-propyl)-thiophene-2-carboxylic acid or 5-(3-(2S-(4-(3-chloro-phenyl)-3R-hydroxy-butyl)-5-oxo-pyrrolidin-1-yl)propyl)-thiophene-2-carboxylic acid, or a pharmaceutically acceptable salt thereof.

8. The method of claim 7 wherein the first compound is 5-(3-{2S-[3R-hydroxy-4-(3-trifluoromethyl-phenyl)-butyl]-5-oxo-pyrrolidin-1-yl}-propyl)-thiophene-2-carboxylic acid or a pharmaceutically acceptable salt thereof.

9. The method of claim 8 wherein the second compound is 17β-estradiol.

10. The method of claim 8 wherein the second compound is conjugated estrogens.

11. The method of claim 1 wherein osteoporosis, osteoporotic fracture, osteotomy, childhood idiopathic bone loss or periodontitis is treated or wherein bone healing following facial reconstruction, maxillary reconstruction or mandibular reconstruction is enhanced, vertebral synostosis is induced, long bone extension is enhanced, the healing rate of a bone graft or a long bone fracture is enhanced or prosthetic ingrowth is enhanced.

12. A method of treating osteoporosis, osteoporotic fracture, osteotomy, childhood idiopathic bone loss or periodontitis or enhancing bone healing following facial reconstruction, maxillary reconstruction or mandibular reconstruction, inducing vertebral synostosis, enhancing long bone extension, enhancing the healing rate of a bone graft or a long bone fracture or enhancing prosthetic ingrowth in a patient in a patient in need thereof, the method comprising continuously administering to the patient in need thereof of a synergistically effective combination of a first compound and a second compound, the first compound being 5-(3-{2S-[3R-hydroxy-4-(3-trifluoromethyl-phenyl)-butyl]-5-oxo-pyrrolidin-1-yl}-propyl)-thiophene-2-carboxylic acid or a pharmaceutically acceptable salt thereof, and the second compound being 17β-estradiol.

13. The method of claim 12 wherein the 5-(3-{2S-[3R-hydroxy-4-(3-trifluoromethyl-phenyl)-butyl]-5-oxo-pyrrolidin-1-yl}-propyl)-thiophene-2-carboxylic acid or a pharmaceutically acceptable salt thereof is continuously administered at a dosage of approximately 0.3 mg/kg/day and the 17β-estradiol is continuously administered at a dosage of approximately 0.01 mg/kg/day.

14. The method of claim 13 wherein the 5-(3-{2S-[3R-Hydroxy-4-(3-trifluoromethyl-phenyl)-butyl]-5-oxo-pyrrolidin-1-yl}-propyl)-thiophene-2-carboxylic acid or a pharmaceutically acceptable salt thereof and 17β-estradiol are continuously administered for a period of at least 28 days.
